SEQUENCE LISTING (28 sequences)

SEQ ID NO: 1 - protein, 11 aa, Artificial Sequence
  Description of Artificial Sequence  Synthetic Peptide
GVAYSMTASAA

SEQ ID NO: 2 - protein, 6 aa, Artificial Sequence
  Description of Artificial Sequence  Synthetic Peptide
EVNLDA

SEQ ID NO: 3 - protein, 6 aa, Artificial Sequence
  Description of Artificial Sequence  Synthetic Peptide
DSGYEV

SEQ ID NO: 4 - protein, 6 aa, Artificial Sequence
  Description of Artificial Sequence  Synthetic Peptide
VVIATV

SEQ ID NO: 5 - DNA, 24 bp, Artificial Sequence
  Description of Artificial Sequence  Synthetic Primer
aagccaccaccacccagacttagg

SEQ ID NO: 6 - DNA, 28 bp, Artificial Sequence
  Description of Artificial Sequence  Synthetic Primer
ctcgagctatggccccggcgctgcgctg

SEQ ID NO: 7 - DNA, 21 bp, Artificial Sequence
  Description of Artificial Sequence  Synthetic Primer
gagggtcctgaggtgctctgg

SEQ ID NO: 8 - DNA, 24 bp, Artificial Sequence
  Description of Artificial Sequence  Synthetic Primer
cctcctcacttcagcagggagatg

SEQ ID NO: 9 - protein, 14 aa, Artificial Sequence
  Description of Artificial Sequence  Synthetic Peptide
GKPIPNPLLGLDST

SEQ ID NO: 10 - protein, 8 aa, Artificial Sequence
  Description of Artificial Sequence  Synthetic Peptide
DYKDDDDK

SEQ ID NO: 11 - protein, 18 aa, Artificial Sequence
  Description of Artificial Sequence  Synthetic Peptide
GMLPADYKDDDDKQGTHL

SEQ ID NO: 12 - protein, 13 aa, Artificial Sequence
  Description of Artificial Sequence  Synthetic Peptide
MLPAQGPRETDEE

SEQ ID NO: 13 - protein, 5 aa, Artificial Sequence
  Description of Artificial Sequence  Synthetic Peptide
TDEST

SEQ ID NO: 14 - protein, 20 aa, Artificial Sequence
  Description of Artificial Sequence  Synthetic Peptide
KTEEISEVNLDAEFRHDSGY

SEQ ID NO: 15 - protein, 24 aa, Artificial Sequence
  Description of Artificial Sequence  Synthetic Peptide
THLGIRLPLRSGLAGPPLGLRLPR

SEQ ID NO: 16 - protein, 5 aa, Artificial Sequence
  Description of Artificial Sequence  Synthetic Peptide
ERTKR

SEQ ID NO: 17 - protein, 20 aa, Artificial Sequence
  Description of Artificial Sequence  Synthetic Peptide
LGLRLPRETDEESEEPGRRG

SEQ ID NO: 18 - protein, 18 aa, Artificial Sequence
  Description of Artificial Sequence  Synthetic Peptide
EIARPDDSLEPFFDSLVK

SEQ ID NO: 19 - protein, 501 aa, Artificial Sequence
  Description of Artificial Sequence  Synthetic Peptide
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 20 - protein, 501 aa, Artificial Sequence
  Description of Artificial Sequence  Synthetic Peptide
MAPALRWLLLWVGSGMLPAQGTHLGIRLPLRSGLAGPPLGLRLPRETDEESEEPGRRGSFVEMVDNLRGKSGQGYYVEMTRGQPLTKLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHIPNIFSLQLCGAGFPLNQTEALASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAVSQSSTGTVMGAVIMEGFYVVFDRARKRIGFAVSACHVHDEFRTAAVEGPFVTADMEDCGYNIPQTDESTLMTIAYVMAAICALFMLPLCLMVCQWRCLRCLRHQHDDFADDISLLK

SEQ ID NO: 21 - protein, 16 aa, Artificial Sequence
  Description of Artificial Sequence  Synthetic Peptide
AQGTHLGIRLPLRSGL

SEQ ID NO: 22 - protein, 12 aa, Artificial Sequence
  Description of Artificial Sequence  Synthetic Peptide
RLPRETDEESEE

SEQ ID NO: 23 - protein, 8 aa, Artificial Sequence
  Description of Artificial Sequence  Synthetic Peptide
DAEFRHDS

SEQ ID NO: 24 - protein, 4 aa, Artificial Sequence
  Description of Artificial Sequence  Synthetic Peptide
SQDD

SEQ ID NO: 25 - protein, 4 aa, Artificial Sequence
  Description of Artificial Sequence  Synthetic Peptide
VVFD

SEQ ID NO: 26 - protein, 4 aa, Artificial Sequence
  Description of Artificial Sequence  Synthetic Peptide
PQTD

SEQ ID NO: 27 - protein, 45 aa, Artificial Sequence
  Description of Artificial Sequence  Synthetic Peptide
QSFRITILPQQYLRPVEDVATSQDDCYKFAVSQSSTGTVMGAVIM

SEQ ID NO: 28 - protein, 44 aa, Artificial Sequence
  Description of Artificial Sequence  Synthetic Peptide
MGAVIMEGFYVVFDRARKRIGFAVSACHVHDEFRTAAVEGPFVT

(19) United States
(12) Patent Application Publication
(10) Pub. No.: US 2004/0180417 A1
(43) Pub. Date: Sep. 16, 2004

(54) Secretase/sheddase with asp-ase activity on the beta-site app-cleaving enzyme (bace, asp2, memepsin2)

(76) Inventors: Nabil G. Seidah, Verdun Quebec (CA); Michel Chretien, Outremont (CA); James A. Cromish, Montreal Quebec (CA)

Correspondence Address: Steven L Highlander, Fulbright & Jaworski, Suite 2400, 600 Congress Avenue, Austin, TX, 78701, US

(21) Appl. No.: 10/343,389

(22) PCT Filed: Aug. 1, 2001
(86) PCT No.: PCT/CA01/01118

(30) Foreign Application Priority Data
Aug. 1, 2000 (CA) 2313828

(51) Int. Cl. A61K 48/00; C12N 9/99; C12N 9/64

(52) U.S. Cl. 435/184; 435/226; 514/44

(57) ABSTRACT

A novel Asp-ase activity, referred to as BACE secretase/sheddase, has been found to cleave the ectoddomain of BACE after Asp₃₇₉ (SQDD↓) and Asp₄₀₇ (VVFD↓), and likely after Asp₄₅₁ (PQTD↓). The cleavage of BACE by BACE secretase/sheddase renders BACE soluble which in turns appears to enhance the generation of the amyloidogenic peptide Aβ, which has been implicated as a major factor in the etiology of Alzheimer's Disease. The current invention concerns the modulation of this novel BACE secretase/sheddase activity for such applications as the prevention or treatment of a neurodegenerative disorder that is characterized by the generation of Aβ protein, including Alzheimer's Disease. The invention further comprises a method for the identification of an agent that can alter the ability of BACE secretase/sheddase to associate with and process a known substrate, a method of determining whether an individual is at risk of developing a neurodegenerative disorder that is characterized by the generation of Aβ protein (such as Alzheimer's Disease) and a kit comprising a vessel or vessels containing BACE secretase/sheddase as well as at least one known substrate of this enzyme, namely, BACE or BACE fragments, or the indirect substrate βAPP.

### FIELD OF THE INVENTION

The present invention relates to β-secretase referred to as the beta-site APP-cleaving enzyme (BACE, Asp2, memepsin 2). More specifically, the present invention concerns a novel Asp-ase that processes BACE, referred to as BACE secretase/sheddase, and the use of this enzyme in the diagnosis, prevention or treatment of neurodegenerative disorders, such as Alzheimer's Disease. The present invention further comprises the use of BACE secretase/sheddase in a screening assay for the identification of agents capable of modifying its activity (modulating agents) as well as the use of BACE secretase/sheddase in a kit.

### BACKGROUND OF THE INVENTION

Alzheimer Disease (AD) is a progressive degenerative disorder of the brain characterized by mental deterioration, memory loss, confusion, and disorientation. Among the cellular mechanisms contributing to this pathology are two types of fibrous protein deposition in the brain: intracellular neurofibrillary tangles composed of polymerized tau protein, and abundant extracellular fibrils comprised largely of β-amyloid (for reviews, see 1-3). Beta-amyloid, also known as Aβ, arises from proteolytic processing of the β-amyloid precursor protein (βAPP) at the β- and γ-secretase cleavage sites. The cellular toxicity and amyloid-forming capacity of the two major forms of Aβ (Aβ₄₀ and especially Aβ₄₂) have been well documented (1-3).

An alternative anti-amyloidogenic cleavage site performed by α-secretase is located within the Aβ peptide sequence of βAPP and thus precludes formation of intact insoluble Aβ. Cleavage by α-secretase within the [HisHisGlnLys↓LeuVal] sequence of KNAPP is the major physiological route of maturation. The products of this reaction are a soluble 100-120 kDa N-terminal fragment (βAPPsα) and a C-terminal membrane-bound ˜9 kDa segment (C83). In several recent reports, metalloproteinases such as ADAM9, 10 and 17 were shown to be involved in the α-secretase cleavage of βAPP (4-6). Enzymes within this family are typically synthesized as inactive zymogens that subsequently undergo prodomain cleavage and activation in the trans Golgi network (TGN). To date, several of the ADAMs have been shown to be activated in a non-autocatalytic manner by other enzymes such as the proprotein convertases (PCs) (7). Thus, it is conceivable that such enzymes may participate in a cascade leading to the activation of α-secretase. In support of this proposal, it has been recently demonstrated that inhibition of PC-like enzymes in HK293 cells by the α1-antitrypsin serpin variant α1-PDX (8) blocks the α-secretase cleavage of βAPP_{SW} (9). Correspondingly, overexpression of a PC (e.g., PC7) increases α-secretase activity. Of the above-mentioned candidate α-secretases, ontogeny and tissue-expression analyses suggest that, in adult human and/or mouse brain neurons, ADAM10 is a more plausible α-secretase than ADAM17 (10).

The amyloidogenic pathway of βAPP processing begins with β-secretase. This enzyme(s) generates the N-terminus of Aβ by cleaving βAPP within the GluValLysMet↓AspAla sequence (SEQ ID NO:1), or by cleaving the Swedish mutant βAPP_{SW} within the GluValAsnLeu↓AspAla sequence (SEQ ID NO:2). In addtion, some cleavage was reported to occur within the Aβ sequence AspSerGlyTyr₁₀↓Glu₁₁Val (SEQ ID NO:3) generating Aβ_{11-40/42} (11). Very recently, five different groups simultaneously reported the isolation and initial characterization of two novel human aspartyl proteinases, BACE (11-15) and its closely related homologue BACE2 (14,15). BACE appears to fulfill all of the criteria of being a β-secretase. While in vitro cleavage specificity analyses of BACE and BACE2 did not reveal clear consensus recognition sequences (11,15) they did lead to the development of novel modified statine inhibitors (13). Comparative modeling of the three-dimensional structure of BACE as a complex with its substrate suggested that BACE would preferentially cleave substrates having a negatively charged residue at P1′ and a hydrophobic residue at P1 (16), which is the case for the β-secretase site in βAPP, βAPP_{SW} and in the generation of the Aβ₁₁₋₄₀ peptide. Both BACE and BACE2 are type-I membrane-bound proteins with a prodomain that, at least for BACE (12), is rapidly cleaved intracellularly. However, little else is known about the mechanism of zymogen processing of these enzymes, including whether their activation is autocatalytic or carried out by other enzymes. Recent data derived from BACE overexpressed in bacteria (15) suggested that zymogen processing of the prosegment's R₄₂LPR45↓ site, which is reminescent of PC-cleavage sites (7), is not autocatalytic; rather it is effected by another proteinase(s). Finally, developmental analysis of the comparative tissue expression of mouse BACE and BACE2 suggested that BACE, but not BACE2, is a good candidate β-secretase in the brain (10).

The second step in the amyloidogenic pathway of βAPP maturation involves cleavages at the γ-secretase sites (ValVal↓IleAla↓ThrVal) (SEQ ID NO:4) to generate either Aβ₄₀ or Aβ₄₂. Recently, in neuronal N2a cells, Aβ₄₀ was shown to be produced within the TGN and subsequently packaged into post-TGN secretory vesicles, suggesting that the TGN is the major intracellular compartment within which the Aβ₄₀-specific γ-secretase is active (17). Although some insoluble, N-terminally truncated Aβₓ₋₄₂ originates in the endoplasmic reticulum (ER), Aβ₄₂ and Aβ₄₀ are formed primarily in the TGN which comprises the major source of the constitutively secreted pool of Aβ that is deposited as extracellular amyloid plaques. Moreover, the generation of either peptide requires that βAPP or its membrane-bound, β-secretase cleavage product C99, passes at least once through endosomal compartments (18). Thus, βAPP trafficking to or retention in particular cellular compartments may critically influence its processing. While the identification of the γ-secretase(s) has not yet been conclusively established (18), some reports have suggested that presenilins are possible candidates (19).

### SUMMARY OF THE INVENTION

In the studies leading to the current invention, PCs were investigated to determine whether they are responsible for the cleavage of the prosegment of BACE, as well as the consequences of blocking this maturation. In addition, several post-translational modifications of BACE and their possible influence on the processing of βAPP and the generation of amyloidogenic Aβ peptides were examined.

The research data obtained indicate that compared to similar levels of expression of full length BACE, expression of a soluble form of beta-APP converting enzyme (BACEₛ), prepared by deleting the transmembrane domain and cytosolic tail, results in a very significant increase in the levels of Aβ peptide produced from the Swedish mutant of APP. In contrast, overexpression of full-length BACE (BACE_{F}) in HK293 cells causes a significant increase in C99. In fact, evidence for BACE C-terminal proteolytic cleavage/shedding is provided, as shown by the detection of apparent 34, 15, 11 and 6 kDa BACE fragments (FIG. 5C, FIG. 7, FIG. 8, FIG. 10, FIG. 11), and BACE shed into the media (FIG. 9). Therefore, BACE_{F} is transformed into C-terminal truncated forms similar to BACE_{S}.

A unique C-terminal proteolytic cleavage of BACE by a novel Asp-ase activity (referred to as BACE secretase/sheddase activity) has been identified. Recent data on the characterization of the nature of BACE C-terminal cleavage reveals this novel BACE secretase/sheddase activity. Since truncation of BACE leads to increased Aβ production, BACE secretase/sheddase is an attractive target to modulate for medicinal and research purposes.

The current invention concerns the modulation of this novel BACE secretase/sheddase activity for such applications as the prevention or treatment of a neurodegenerative disorder that is characterized by the generation of Aβ protein, including Alzheimer's Disease. The invention further comprises a method for the identification of an agent that can alter the ability of BACE secretase/sheddase to associate with and process a known substrate, a method of determining whether an individual is at risk of developing a neurodegenerative disorder that is characterized by the generation of Aβ protein (such as Alzheimer's Disease) and a kit comprising a vessel or vessels containing BACE secretase/sheddase as well as at least one known substrate of this enzyme, namely, BACE or BACE fragments, or the indirect substrate βAPP.

An object of the present invention is therefore the inhibition of Aβ plaque formation in such neurodegenerative disorders as Alzheimer's Disease through the modulation of the newly-identified BACE secretase/sheddase activity in order to treat and/or prevent the progression of this disease.

A further object of the present invention is to make use of the newly-identified BACE secretase/sheddase activity in a screening assay, in a diagnostic assay for neurodegenerative disorders characterized by the generation of Aβ protein (such as Alzheimer's Disease) and in a kit.

### DESCRIPTION OF THE DRAWINGS

FIG. 1: HK293 cells were transiently co-transfected with either ([BACE_{F}]_{FG/V5}+BDNF) [control, CTL] (A,C) or ([BACE_{F}]_{FG/V5}+α1-PDX) (B,D) cDNAs. Two days post-transfections the cells were pulse-labeled in the absence or presence of 5 mM BFA for 15 min with [³⁵S]Met and then chased for 1 or 2h. Cell lysates were immunoprecipitated with either the FG or V5 mAbs and analysed by SDS-PAGE on 8% tricine gels. The migration position of the 53 kDa molecular mass standard and those of proBACE (pBACE) and BACE are emphasized.

FIG. 2: [A] HK293 cells were transiently co-transfected with cDNAs coding for either ([BACE_{F}]_{FG/V5}+BDNF) [control, CTL], ([BACE_{F}-R45A]_{FG/V5}+BDNF) or ([BACE_{F}-R42A]_{FG/V5}+BDNF) or ([BACE_{F}]_{FG/V5}+either α1-PDX, the prosegments of furin, PC5, PC7, SKI-1, furin-mutated (α2M-F) or wild type (α2M) α2-macroglobulin. The cells were pulse-labeled for 20 min with [³⁵S]Met and then chased for 90 min. Cell lysates were immunoprecipitated with the FG mAb and analysed by SDS-PAGE on 8% tricine gels. [B] HK293 cells were transiently co-transfected with cDNAs coding for either ([BACE_{F}]_{FG/V5}+BDNF) [CTL], ([BACE_{F}]_{FG/V5}+furin) or ([BACE_{F}]FG/V5+α1-PDX). The cells were then pulse-labeled for 2h with Na₂[³⁵SO₄]. Cell lysates were immunoprecipitated with the FG or V5 mAbs and analysed by SDS-PAGE on 8% tricine gels. (The higher apparent size of BACE_{G} in the CTL lane compared to the furin lane is due to end-lane distortion.) The migration positions of those proBACE in the ER (pBACE_{ER}) or Golgi (pBACE_{G}) are emphasized.

FIG. 3: Western blot analysis of 1-4 h in vitro processing of wild type (WT) [proBACE_{S}]_{FG/V5} or the (R45A) mutant [proBACE_{S}-R45A]_{FG/V5} by either furin, PC5-A, PACE4 or PC7 in the absence or presence of 1 μM of PC-prosegments (pPCs). Flag-M2 (FG) or V5-HRP monoclonal antibodies were used.

FIG. 4: [A] HK293 cells were transiently transfected with cDNAs coding for either [BACE_{F}]_{FG}, [BACE_{F}-Δp]_{FG} or [BACE_{S}]_{V5}. The cells were pulse-labeled for 20 min (−) with [³⁵S]Met and then chased for 1 h or 2h. Cell lysates and media (for BACE_{S}) were immunoprecipitated with the FG or V5 mAbs and analysed by SDS-PAGE on 8% tricine gels. [B] HYK293 cells were transiently transfected with [BACE_{S}]_{V5} cDNA. The cells were then pulse-labeled for 2h with Na₂[³⁵SO₄]. Cell lysates were immunoprecipitated with the V5 mAb. Equal aliquots of SDS-PAGE-purified proteins were then digested overnight at 37° C. with 5 mU of either endoH or endoF (Glyko Inc.) or 80 mU of arylsulfatase (ASase; Sigma). The products were analysed by SDS-PAGE on 8% tricine gels. [C] HK293 cells were transiently transfected with cDNAs coding for either [BACE_{F}]_{FG}, [BACE_{F}-C482,485A]_{FG}, [BACE_{F}-C478,482,485A]_{FG}, [BACE_{F}-Δp]_{FG} or [BACE_{S}]_{V5}. The cells were pulse-labeled for 2h with [³H]palmitic acid. Cell lysates were immunoprecipitated with FG or V5 (for BACE_{S}) mAbs and analysed by SDS-PAGE on 8% tricine gels.

FIG. 5: HK293 cells were transiently transfected with cDNAs coding for either [A,B] (BDNF+βAPP_{SW}) [CTL] or ([BACE_{F}]_{FG}+βAPP_{SW}), [C] [BACE_{F}]_{FG} or [BACE_{S}]_{FG}. The cells were pulse-labeled for 3h with [³⁵S]Met at either 37° C. in the absence or presence of 90 μM BFA or 250 nM bafilomycin or at 20° C. Cell lysates were immunoprecipitated with either [A] the FG mAb or [B] the 1-16 Aβ antibody, and analysed by SDS-PAGE on 8% tricine gels. [C] FG antibody, and analysed by SDS-PAGE on 8% tricine gels. The arrowhead point to an ˜6 kDa intracellular stub of BACE_{F}.

FIG. 6: HK293 cells were transiently co-transfected with cDNAs coding for (βAPP_{SW}+BDNF) [−], or βAPP_{SW} together with either [BACE_{S}]_{V5}, [BACE_{F}]_{FG}, [BACE_{F}-D93A]_{FG}, [BACE_{F}-R45A]_{FG}, or [BACE_{F}-Δp]_{FG}. The cells were pulse-labeled for 3 h with [³⁵S]Met. The cell lysates [A] or media [B,C] were immunoprecipitated [A,C] with the 1-16 Aβ antibody, and in [B] with the 1-40 Aβ antibody (A8326), and analysed by SDS-PAGE on 8% [A,C] or 14% [B] tricine gels. The migration positions of C99, Aβ, Aβₓ₋₄₀ APP_{S} and Aβ₁₇₋₄₀ known as p3 (generated by α- and γ-secretases) are shown.

FIG. 7: HK293 cells were transiently transfected with cDNAs coding for either [BACE_{F}]_{FG} or an empty pIRES vector [control, CTL]. Following a 4 hr pulse with ³⁵S-Met cell lysates were immunoprecipitated with FG antibodies, denatured in the presence [reduced] or absense [non-reduced] of 2-mercaptoethanol and subsequently analysed by SDS-PAGE on 8% tricine gels. The arrow heads point to apparent BACE_{F} cleavage products of 34, 15, 11 and 6 kDa. The exposure time was 8 hours.

FIG. 8: [A] Neuro 2a APP_{SW} cells were transiently transfected with cDNA for [BACE_{F}]_{FG}. Cells were labeled with ³⁵S-Met for 3 hrs in the absence (−, DMSO control) or presence of 100 uM of a substrate based γ-secretase inhibitor (+γ-sec I, DFK-167 Enzyme Systems products). Cell lysates were immunoprecipitaed with FG antibodies, reduced and analyzed by SDS-PAGE on 8% tricine gels. Cell lysates [B] and media [C] were immunoprecipited with antibody APP711-03 and analyzed by SDS-PAGE on 8% tricine gels. [D] Media was immunoprecipited with the 1-40 Aβ antibody and analyzed on a 14% tricine gel. The exposure time was 3 days.

FIG. 9: Neuro 2a APP_{SW} cells were transiently transfected with cDNAs for [BACE_{F}]_{FG}, [BACE_{S}]_{V5}, or the pIRES control [CTL]. Media and cells were analyzed by immunoprecipitation with an antibody to BACE (BACE 41—Research Genetics, described in Materials and Methods) following a 3 hr chase with ³⁵S-Met. The SDS-PAGE 8% tricine gels were exposed to film for 5 hrs. The positions of BACE_{S} in the media, and the cellular 34 and 15 kDa bands are indicated.

FIG. 10: HK293 cells were transiently transfected with cDNA for [BACE_{F}]_{FG}. Cells were labeled with ³⁵S-Met or ³H-Phenylalanine for 3 hrs as indicated. Following immunoprecipitation with FG antibodies, the 15 kDa BACE fragment (see FIG. 7) was purified by preparative SDS-PAGE and extracted. Radiosequencing was performed as described under Materials and Methods. The amino acid sequence of BACE starting at Gln₃₅₅ and encompassing the N-terminus of the 15 kDa BACE fragment is shown (SEQ D NO: 27).

FIG. 11: HK293 cells were transiently transfected with cDNA for [BACE_{F}]_{FG}. Cells were labeled with ³H-phenylalanine for 3 hrs as indicated. Following immunoprecipitation with FG antibodies, the 11 kDa BACE fragment (see FIG. 7) was purified by preparative SDS-PAGE, extracted and radiosequencing was performed. The amino acid sequence of BACE starting at MeT₃₉₄ and encompassing the N-terminus of the 11 kDa BACE fragment is shown (SEQ ID NO: 28).

### DETAILED DESCRIPTION

In order to provide a clear and consistent understanding of terms used in the present description, a number of definitions are provided hereinbelow.

Unless defined otherwise, the scientific and technological terms and nomenclature used herein have the same meaning as commonly understood by a person of ordinary skill to which tis invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain.

As used herein, the term “BACE fragments” refers to stretches of BACE amino acid sequence that contain the BACE secretase/sheddase cleavage sites defined more particularly below.

As used herein, agonists and antagonists of BACE sheddase/secretase interaction (discussed further below) also include potentiators of known compounds with such agonist or antagonist properties. In one embodiment, agonists can be detected by contacting the indicator cell with a compound or mixture or library of molecules for a fixed period of time is then determined.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, neuronal cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

For administration to humans, the prescribing medical professional will ultimately determine the appropriate form and dosage for a given patient, and this can be expected to vary according to the chosen therapeutic regimen (e.g. DNA construct, protein, cells), the response and condition of the patient as well as the severity of the disease.

Composition within the scope of the present invention should contain the active agent (e.g. fusion protein, nucleic acid, and molecule) in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

The invention provides efficient methods of identifying pharmacological agents or lead compounds for agents capable of mimicking or modulating BACE secretase/sheddase function and preventing the production of the AP peptide. Identified reagents find use in the pharmaceutical industries for animal and human trials; for example, the reagents may be derived and rescreened using ill vitro and in vivo assays to optimize activity and minimize toxicity for pharmaceutical development.

Agents that could be used to manipulate the function of BACE secretase/sheddase include specific antibodies that can be modified to a monovalent form, such as Fab, Fab′, or Fv, specifically binding oligopeptides or oligonucleotides and most preferably, small molecular weight organic receptor agonists. See, Harlow and Lane (1988) Antibodies, A Laboratory Manual, Cold Spring Harbor Laboratory, for general methods. Anti-idiotypic antibody, especially internal imaging anti-ids are also prepared using the disclosures herein.

Other prospective BACE secretase/sheddase specific agents are screened from large libraries of synthetic or natural compounds. For example, numerous means are available for random and directed synthesis of saccharide, peptide, and nucleic acid based compounds. Alternatively, libraries of natural compounds in the form of bacterial, fungal, plant and animal extracts are available or readily producible. Additionally, natural and synthetically produced libraries and compounds are readily modified through conventional chemical, physical, and biochemical means. See, e.g. Houghten et al. and Lam et al (1991) Nature 354, 84 and 81, respectively, and Blake and Litzi-Davis (1992), Bioconjugate Chem 3, 510.

The utility of agents affecting BACE secretase/sheddase function are identified with assays employing the lead compound of interest and testing its effect on Aβ production either in the absence or the presence of βAPP. For example, a method for identifying an agent that can alter the ability of BACE secretase/sheddase to associate with and process a known substrate might comprise the following:

in a reaction mixture, allowing BACE secretase/sheddase to bind to a known substrate of BACE secretase/sheddase in the presence of an agent to be tested; and

measuring the production of BACE C-terminal cleavage products, shed BACE or Aβ in the presence of the agent to be tested, and comparing same under conditions when the agent to be tested is absent from the reaction mixture.

The method relies on the activity of BACE secretase/sheddase in the presence of at least one direct substrate for this enzyme, namely BACE or BACE fragments, or in the presence of the indirect substract βAPP. (βAPP is considered an indirect substrate for BACE secretase/sheddase for the following reason: BACE secretase/sheddase reacts with BACE or BACE fragments and, if either one of these substrates is suitably modified, it can then react with βAPP to generate the amyloidogenic Aβ peptide.)

Useful agents are typically those that bind to and modulate BACE secretase/sheddase function, such as those that inactivate either enzyme and prevent the formation of Aβ. Preferred agents are receptor-specific and do not cross react with other neural or lymphoid cell membrane proteins. Useful agents may be found within numerous chemical classes, though typically they are organic compounds and preferably, small organic compounds. Small organic compounds have a molecular weight of more than 150 yet less than about 4,500, preferably less than about 1500, more preferably, less than about 500. Exemplary classes include peptides, saccharides, steroids, heterocyclics, polycyclics, substituted aromatic compounds, and the like.

Selected agents may be modified to enhance efficacy, stability, pharmaceutical compatibility, and the like. Structural identification of an agent may be used to identify, generate, or screen additional agents. For example, where peptide agents are identified, they may be modified in a variety of ways as described above, e.g. to enhance their proteolytic stability. Other methods of stabilization may include encapsulation, for example, in liposomes, etc. The subject binding agents are prepared in any convenient way known to those skilled in the art.

For therapeutic uses, agents affecting BACE secretase/sheddase function may be administered by any convenient way. Small organics are preferably administered orally; other compositions and agents are preferably administered parenterally, conveniently in a pharmaceutically or physiologically acceptable carrier, e.g., phosphate buffered saline, or the like. Typically, the compositions are added to a retained physiological fluid such as blood or synovial fluid. For CNS administration, a variety of techniques are available for promoting transfer of the therapeutic across the blood-brain barrier including disruption by surgery or injection, drugs which transiently open adhesion contact between CNS vasculature endothelial cells, and compounds which facilitate translocation through such cells.

As examples, many such therapeutics are amenable to direct injection or infusion, topical, intratracheal/nasal administration e.g. through aerosal, intraocularly, or within/on implants (such as collagen, osmotic pumps, grafts comprising appropriately transformed cells, etc.). A particularly useful application involves coating, imbedding or derivatizing fibers, such as collagen fibers, protein polymers, etc. with therapeutic peptides. Other useful approaches are described in Otto et at. (1989) J Neuroscience Research 22, 83-91 and Otto and Unsicker (1990) J Neuroscience 10, 1912-1921. Generally, the amount administered will be empirically determined, typically in the range of about 10 to 1000 μg/kg of the recipient. For peptide agents, the concentration will generally be in the range of about 50 to 500 μg/ml in the dose administered. Other additives may be included, such as stabilizers, bactericides, etc. These additives will be present in conventional amounts.

For antisense applications where the inhibition of expression is indicated, especially useful oligonucleotides are between about 10 and 30 nucleotides in length and include sequences surrounding the disclosed ATG start site, especially the oligonucleotides defined by the disclosed sequence beginning about 5 nucleotides before the start site and ending about 10 nucleotides after the disclosed start site.

The compositions and methods disclosed herein may be used to effect gene therapy. See, e.g. Zhu et al. (1993) Science 261, 209-211; Guiterrez et al. (1992) Lancet 339, 715-721. For example, cells are transfected with sequences encoding a peptide or ribozyme operably linked to gene regulatory sequences capable of effecting altered BACE secretase/sheddase expression, regulation, or function. To modulate BACE secretase/sheddase expression, regulation, or function, target cells may be transfected with complementary antisense polynucleotides. For gene therapy involving the grafting/implanting/transfusion of transfected cells, administration will depend on a number of variables that are ascertained empirically. For example, the number of cells will vary depending on the stability of the transfered cells. Transfer media is typically a buffered saline solution or other pharmacologically acceptable solution. Similarly the amount of other administered compositions (e.g. transfected nucleic acid, protein, etc.) will depend on the manner of administration, purpose of the therapy, and the like.

The present invention farther comprises a method for determining whether an individual is at risk of developing a neurodegenerative disorder that is characterized by the generation of Aβ protein, such as Alzheimer's Disease. Generally, this method involves extracting a sample tissue or fluid (such as cerebrospinal fluid or blood platelets) from the individual and determining whether the level of BACE C-terminal cleavage products, shed BACE or Aβ protein in the tissue or fluid sample is higher than the level in a tissue or fluid sample from a healthy subject, as an indication that the individual is at risk for the neurodegenerative disorder. The method relies on the activity of BACE secretase/sheddase in the presence of at least one direct substrate for this enzyme, namely BACE or BACE fragments, or in the presence of the indirect substract βAPP. βAPP is considered an indirect substrate for BACE secretase/sheddase for the following reason: BACE secretase/sheddase reacts with BACE or BACE fragments and, if either one of these substrates is suitably modified, it can then react with βAPP to generate the amyloidogenic Aβ peptide.)

The present additionally comprises a kit that is suitable for such diagnoses. For example, a compartmentalized kit in accordance with the present invention includes any kit in which reagents are contained in separate containers or vessels. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (fluid or tissue) and containers with BACE secretase/sheddase and at least one substrate of this enzyme, namely, BACE or BACE fragments, or the indirect substrate βAPP.

Materials and Methods

Mouse BACE and its mutants—Full length mouse BACE (mBACE_{F}) was cloned from AtT20 cells by RT-PCR (Titan One-Tube, Boehringer) using the following nested sense (S) and antisense (AS) oligonucleotides: S1=AAGCCACCACCACCCAGACTTAGG (SEQ ID NO:5); S2=CTCGAGCTATGGCCCCGGCGCTGCGCTG (Xho-I site at 5′) (SEQ ID NO:6) and AS1=GAGGGTCCTGAGGTGCTCTGG (SEQ ID NO:7); AS2=CCTCCTCACTTCAGCAGGGAGATG (SEQ ID NO:8). The final product (1519 bp) was then completely sequenced and matched with the published structure (11), then subcloned into the expression vector pcDNA3.1/Zeo (Invitrogen). In order to detect recombinant BACE_{F}, either a V5 (GKPIPNPLLGLDST (SEQ ID NO:9); [BACE_{F}]_{V5}) or Flag (DYKDDDDK (SEQ ID NO:10) were added, in phase, by PCR; [BACE_{F}]_{FG}) epitope to the C-terminal amino acid of the cytosolic tail of mouse BACE. A BACE_{F} contruct was also prepared in pIRES2-EGFP (Invitrogen) in which a FLAG epitope was introduced just after the signal peptide cleavage site (giving the sequence . . . GMLPA↓DYKDDDDK-QGTHL . . . ) (SEQ ID NO:11) and a V5 epitope was at the C-terminus of the molecule [BACE_{F}]_{FG/V5}. Other BACE constructs were also prepared including: (1) an active site D93A mutant singly [BACEF-D93A]FG or doubly tagged [BACE_{F}-D93A]_{FG/V5}; (2) a prosegment deletion mutant [BACE_{F}-Δp]_{FG} in which the signal peptide ending at Ala₁₉ is fused directly to the sequence . . . . MLPA₁₉↓QG-PRE₄₆TDEE . . . (SEQ ID NO:12); (3) PC-cleavage site (R₂LPR₄₅) mutants [BACE_{F}-R45A]FG as well as the double tagged [BACE_{F}-R42A]_{FG/V5} and [BACE_{F}-R45A]_{FG/V5}; (4) deletion of the prosegment in the active site mutant [BACE_{F}-Δp-D93A]_{FG}; and (5) cytosolic tail Cys-mutants, including single [BACE_{F}-C478A]_{FG}, [BACE_{F}-C482A]_{FG}, [BACE_{F}-C485A]_{FG}, double [BACE_{F}-C482,485A]_{FG}, and triple [BACE_{F}-C478,482,485A]_{FG} mutants. Soluble forms of BACE (BACE_{S}) were also prepared by deleting the transmembrane domain (TMD) and cytosolic tail (CT), leaving the sequence . . . TDEST₄₅₄ (SEQ ID NO:13) followed by a V5 epitope. These constructs included [BACE_{S}]_{V5}, [BACE_{S}]_{FG/V5}, [BACE_{S}-R42A]_{FG/V5} and [BACE_{S}-R45A]_{FG/V5}.

Transfections and biosynthetic analyses—All transfections were done on 2−4×10⁵ HK293 cells using Effectene (Qiagen) and a total of 1-1.5 μg of BACE contruct cDNAs subcloned into the vector pIRES2-EGFP. Two days post-transfection the cells were washed and then pulse-incubated for various times with either 200 μCi/ml of [³⁵S]Met; 400 μCi/ml Na₂[³⁵SO_{4], [}³H]Leu, [³H]Arg, [³H]Ser; or 1 mCi/ml [³H]palmitate EN) (20). Pulse-chase experiments with [³⁵S]Met were carried out as previously described (21). Cells were lysed in immunoprecipitation buffer [150 mM NaCl, 50 mM Tris-HCl pH 6.8, 0.5% NP40, 0.5% sodium deoxycholate and a protease inhibitor cocktail (Roche Diagnostics). The lysates and media were then prepared for immunoprecipitations as reported (22). The monoclonal antibodies used were directed against either the FL (Flag-M2; 1:500 dilution; Stratagene) or V5 (1:1000 dilution; Invitrogen) epitopes. Rabbit polyclonal antisera included those directed against aa 1-16 of human Aβ (produced in laboratory, dilution 1:200); anti-β-amyloid, recognizing mostly the C-terminal part of Aβ40 (A8326, dilution 1:200, Sigma); and FCA 18, recognizing all peptides starting with the Asp at the N-terminus of Aβ (23). Immunoprecipitates were resolved on SDS-PAGE (either 8% or 14% tricine gels) and autoradiographed (21). All PC inhibitor proteins were cloned in pcDNA3 (Invitrogen), including those of α1-PDX (8); the preprosegments of furin, PC7 (24), PC5 (25), SKI-1 (26,27); and wild type (α2-M) and furin-site mutated (α2-MG-F) α2-macroglubulin (28).

In vitro assays and Western blotting—Enzymatically active BACE was obtained from 10-20 fold-concentrated media of HK293 cells transiently transfected with the cDNAs of [BACE_{S}]_{FG/V5}, [BACE_{S}-R42A]_{FG/V5} or [BACE_{S}-R45A]_{FG/V5}. Beta-secretase activity was evaluated using a 20 aa synthetic peptide spanning the cleavage site (KTEEISEVNL↓DAEPRHDSGY) (SEQ ID NO:14) of βAPP_{SW}. Reactions were carried out using 10-30 μM peptide for 16-18 hrs at 37° C. in 100 μl of 50 mM NaOAc (pH 4.5), plus 10 μg/ml of leupeptin to inhibit low levels of a non-β-secretase proteolytic activity. The digestion products, separated and quantitated via RP-HPLC TFA/acetonitrile gradient) on a C-18 column (Vydak), were identified using MALDI-TOF mass spectroscopy (Voyager/Perkin Elmer). ProBACE incubations were carried out in the same fashion using either [proBACE_{S}]_{FG/V5} or [proBACEs-R42A]_{FGN/V5} purified on an anti-FL M1 agarose affinity column (Sigma) according to the manufacturer's instructions. Incubations with the peptide comprising the entire prosegment of mBACE (THLGIRLPLRSGLAGPPLGLRLPR (SEQ ID NO:15), 10-30 μM final concentration) were carried out as for P-secretase activity measurements.

PC-mediated digestions entailed preincubating the various BACE constructs for up to 4 h in 50 J11 of 50 mM Tris-Oac (pH 7.0) plus 2 mM CaCl₂ (and 0.1% Triton X-100 (v/v), for Western blot analysis of BACE prosegment removal) in the presence of media from BSC40 infected with vaccinia virus recombinants of human furin, PACE4, and mouse PC5-A (29), as well as rat PC7 (30). The activities of the different PC preparations were estimated according to the initial hydrolysis rates of the pentapeptide fluorogenic substrate pERTKR-MCA (SEQ ID NO:16) (29,30). PC activity-inhibited controls comprised 4 h incubations in the presence of 1 μM of the corresponding purified prosegments of PCs (24,25). Digestions of the PC cleavage site-spanning peptide (LGLRLPR↓ETDEESEEPGRRG) (SEQ ID NO:17) by PCs were carried out as above for the BACE preincubations (except in 100 μL), whereas digestions by BACE were as for β-secretase activity at pH 4.5 or 6.5. Digestion products were again quantitated by RP-HPLC and MALDI-TOF mass spectroscopic analysis.

Western blot analyses of the reaction products were carried out following 10% SDS-PAGE using either the FG (1:1000 dilution) or V5-HRP (1:5000 dilution) monoclonal antibodies (Stratagene). The secondary antibody for FG consisted of anti-mouse HRP-coupled IgGs (Boehringer Mannheim).

Generation of antiserum to human BACE—Monospecific polyclonal rabbit antiserum that recognizes the peptide sequence EIARPDDSLEPFFDSLVK (SEQ ID NO:18) in human (NCBI Protein NP_{—}036236) (SEQ ID NO:19) and mouse BACE (NCBI P56818) (SEQ ID NO:20) was generated by Research Genetics. The initial immunogen was a 393 long fragment of human BACE (from MVDNLRG to OTDESTL) expressed as a C-terminal His-tagged protein in a pET-24B vector in bacteria BL21 (DE3)pLysS (Stratagene).

Radiosequencing of 15, 11, 34 and 6 kDa BACE fragments—The SDS PAGE extracted fragments were treated to remove excess salts and SDS and applied on a PVDF membrane into an ABI Procise 477 cLC sequencer. The standard program was modified for radioactive sequencing, whereby the effluent was directed to a fractio collector. Typically, 20-20 sequencer cycles were collected for each run. Subsequently, the radioactive counts were quantified on a Beckman sequencer.

Results

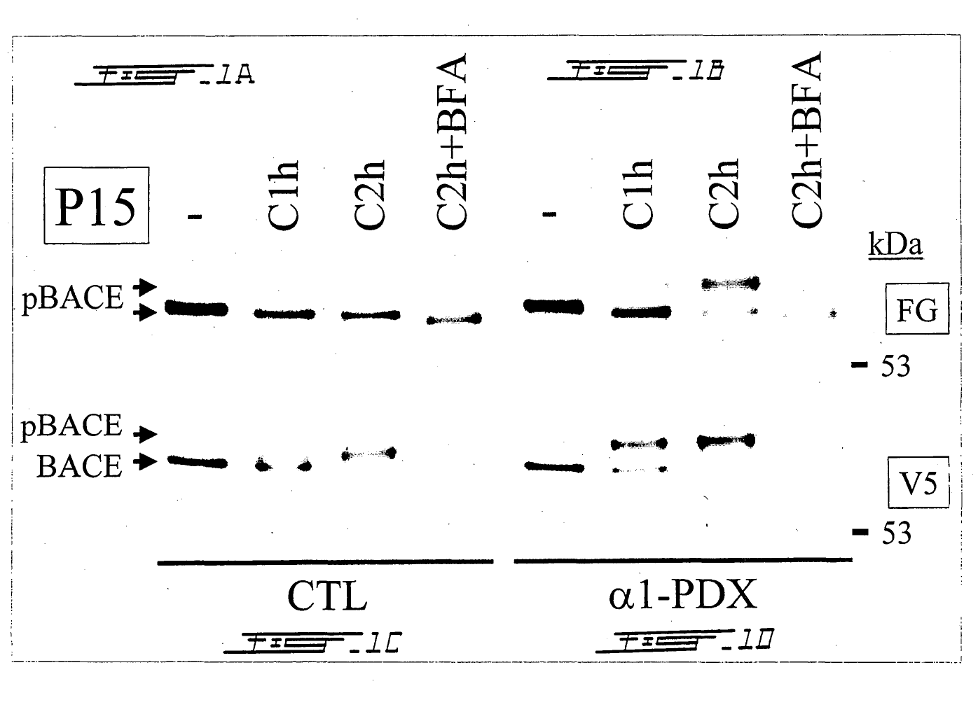

Biosynthesis and processing of BACE—In order to characterize the biosynthetic pathway of BACE and its post-translational modifications, the enzyme from the mouse corticotroph cell line AtT20 was cloned. The resultant, fully sequenced 1519 bp product corresponded to the published mouse sequence (11). In order to detect membrane bound proBACE or BACE, the V5 epitope at the C-terminus of the cytosolic tail was used. Alternatively, the N-terminal Flag epitope (FG) immediately following the signal peptidase cleavage site to specifically detect proBACE was employed. This doubly-tagged, full-length (F) protein [BACE_{F}]_{FG/V5} was co-expressed in human kidney epithelial cells C1K293) either with a control (CTL) [brain derived neurotrophic factor (BDNF)] or α1-PDX cDNA. Two days after transfection, the cells were pulse-labeled with [³⁵S]Met for 15 min (P15). They were then chased for 1 h or 2h in the presence or absence of the fungal metabolite brefeldin A (BFA), which promotes fusion of the cis, medial and trans Golgi (but not the TGN) with the ER (31). Cell extracts were immunoprecipitated with either FG or V5 monoclonal antibodies and analysed by SDS-PAGE (FIG. 1). In the absence of BFA and α1-PDX at P15 (FIG. 1A), the FG epitope reveals a 66 kDa proBACE form that is gradually transformed first into a 64 kDa (C1h) and then into a minor 72 kDa (C2h) proBACE form. Whereas the 72 kDa form is not apparent in the presence of BFA (the major band is visible at 63 kDa), it is greatly enriched in the presence of α1-PDX (FIG. 1B). Treatment with endoglycosidases revealed that the 63 and 64 kDa proBACE forms are sensitive to both endoH and endoF, whereas the 72 kDa form is sensitive only to endoF (not shown). These data suggest that the 63 and 64 kDa bands represent immature (likely ER-resident), N-glycosylated proBACE whereas the 72 kDa form represents mature proBACE. Only in the presence of α1-PDX does proBACE immunoreactivity accumulate in the Golgi apparatus. In immunoprecipitation experiments employing the V5 epitope, the 2h-chase period revealed mainly a 68 kDa band (FIG. 1C). In the presence of α1-PDX (FIG. 1D), an accumulation of a 72 kDa protein reminiscent of proBACE (FIG. 1C) was observed.

N-terminal radiosequencing (26,30) was carried out on SDS-PAGE-purified immunoprecipitates. The C-terminally flagged 72 kDa [proBACE_{F}]_{FG}, labeled with [³H]Leu and produced in the presence of α1-PDX, had a Leu_{3,7,9,13} sequence (not shown). This is consistent with the protein starting at Thr₂₂ (AQG₂₁↓HLGIRLPLRSGL) (SEQ ID NO:21) which is just after the signal peptidase cleavage site (8,9). The corresponding 68 kDa protein, labeled with [³H]Ser, revealed a Ser₆ signal (not shown), compatible with the protein being mature BACE obtained following removal of the prosegment (aa 22-45) at the RLPR₄₅↓E₄₆TDEESEE sequence (SEQ ID NO:22).

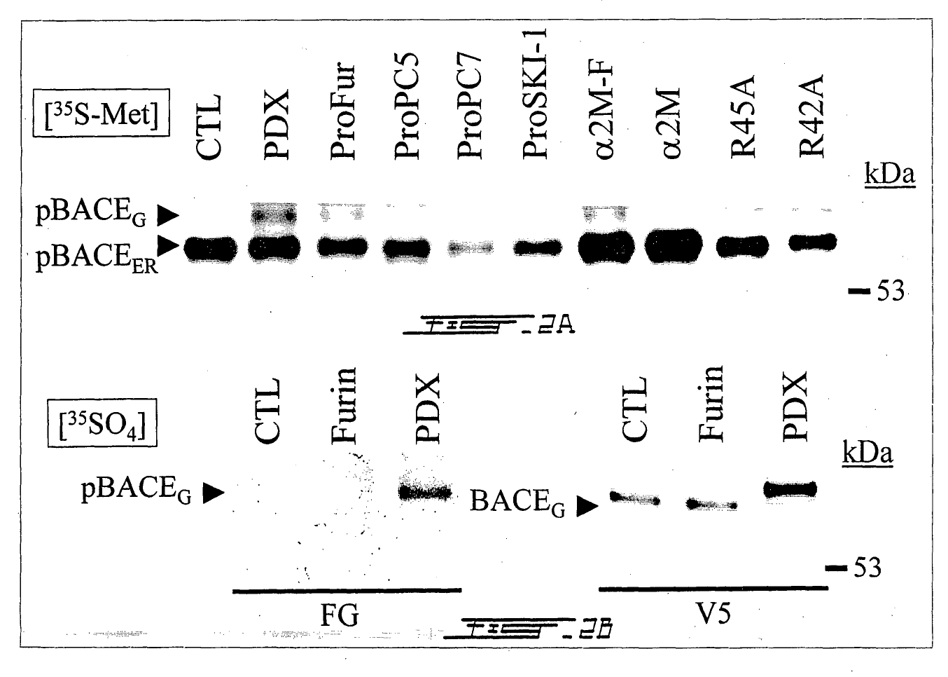

In order to determine whether a proprotein convertase(s) could carry out the processing of proBACE to BACE, the doubly-tagged [BACE_{F}]_{FG/V5} was transiently co-expressed in HK293 cells with an array of PC-inhibitors including: α1-PDX (8,21); the pre-prosegments of furin, PC7 (24), PC5 (25), and SKI-1 (27); and the wild type (α2M) and furin-inhibiting mutant (α2M-F) forms of α2-macroglubulin (28). In addition, mutant forms of BACE were prepared in which the PC-consensus cleavage site Arg residues in the prosegment were replaced by Ala at positions 42 or 45 (R42A or R45A, respectively). The transfected cells were pulse-labeled for 20 min with [³⁵S]Met and then chased for 90 min without label. Following immunoprecipitation of the cell lysates with a FG antibody, the material was analysed by SDS-PAGE. When BACE was co-expressed with either α1-PDX, proFur, proPC5 or α2M-F, the quantity of the 72 kDa proBACE (pBACE_{G}, Golgi form) was elevated (FIG. 2A). Similar results were seen for both the R42A or R45A prosegment cleavage site mutants. In contrast, the 72 kDa proBACE was barely detectable in the control, proPC7, proSKI-1 or α2M co-expressions. Parallel control experiments (not shown) verified that the prosegments of PC7 (24) and SKI-I (27) were able to inhibit processing of appropriate substrates by their cognate enzymes. These data strongly support the hypothesis that a PC-like enzyme may be involved in the processing of proBACE into BACE. The prosegment results implicate furin and PC5 as likely PC candidates, whereas PC7 and SKI-1 appear unlikely to mediate this process. The finding that the Arg residues at the predicted canonical R₄₂-X-X-R₄₅↓ site are essential for proBACE processing is also consistent with the reported cleavage specificities of furin and PC5 (7).

In order to better define the region of the Golgi where proBACE processing occurs, [BACE_{F}]_{FG/V5} was co-expressed in HK293 cells with either furin or α1-PDX and then labeled the cells for 2 h with Na₂[³⁵SO₄]. SDS-PAGE analyses of the FG or V5-immunoprecipitates are shown in FIG. 2B. Using the FG-antibody, it was observed that proBACE is weakly sulfated (CTL). In the presence of α1-PDX, the intensity of the 72 kDa [³⁵SO₄]-proBACE (pBACE_{G}) was greatly enhanced. The V5-immunoprecipitates clearly demonstrated that BACE is sulfated, and further revealed that furin digestion appears to lower the average apparent mass of sulfated BACE from 72 (pBACE_{G}) to 68 kDa (BACE_{G}). Finally, the data suggest that processing of proBACE by a PC-like enzyme into BACE occurs at the TGN or in a subsequent compartment. Not only are sulfotransferases located in this region of the secretory pathway (32,33) but, with the exception of PC5-B (34), all other PCs become active only at or beyond the TGN (7), which is also a major site where α1-PDX acts (21).

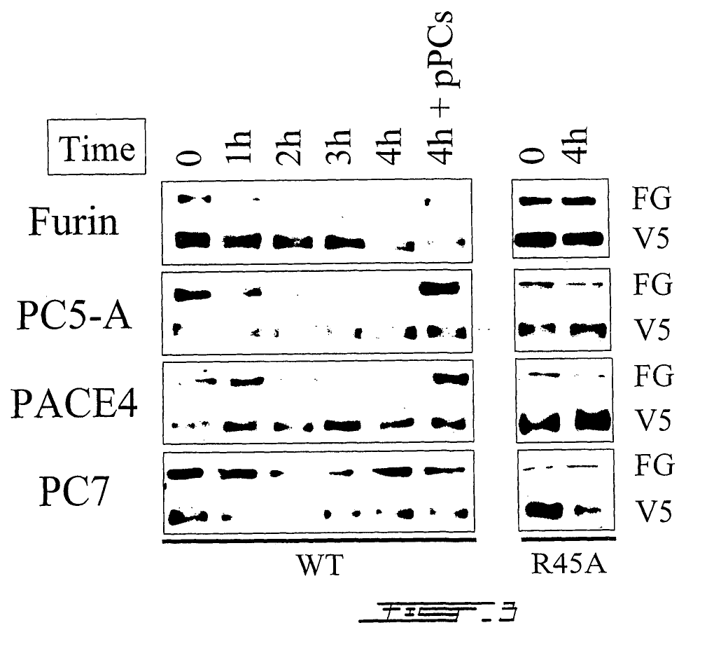

The next set of experiments were devised to determine whether PCs could process proBACE in vitro. In preliminary work, testing was conducted to find which of the PCs expected to be active in the constitutive secretory pathway could correctly cleave a peptide (proBACE 38-54) spanning the N-terminal furin-concensus site. The best processing rates were observed with furin and PC5 (not shown), followed distantly by PACE4. PC7 could barely cleave this sequence, even when a 10-fold excess (as assessed by pERTKR-MCA hydrolysis) of activity was employed. At the same time, no detectable cleavage of this peptide was observed by either crude or partially purified soluble BACE [BACE_{S}]_{V5} (not shown), lending further support to the view that the BACE does not autocatalytically remove its own propeptide. The PC-mediated processing of a doubly tagged soluble (S) form of proBACE [BACE_{S}]_{FG/V5} expressed in HK293 cells was examined next. Western blots of the secreted enzyme probed by the FG antibody revealed that some of the enzyme was still in the form of proBACE_{S}. The concentrated medium of HK293 cells was thus used as a source of proBACE_{S}. Aliquots of this medium (equalized by their V5 immunoreactivities) were incubated with equivalent hydrolytic activities (estimated using the fluorogenic substrate pERTKR-MCA) of partially purified furin, PC5, PACE4 and PC7 for 1-4 hours. The digestion products were then run on SDS-PAGE and revealed by western blotting using either the FG or V5 antibodies. The data demonstrated that furin could completely process proBACE into BACE within 2h, which was superior to the abilities of PC5 and PACE4 to carry out this cleavage (FIG. 3). In contrast, PC7 is barely, if at all, able to perform this reaction. As further confirmation of the identity of the enzyme(s) carrying out this conversion, we treated the 4h proBACE digestion reaction with 1 μM purified PC prosegments (pPCs) produced in bacteria as previously reported (24). Correspondingly, the pPCs of furin, PC5 and PACE4 inhibited proBACE processing. Finally, analysis of the R45A mutant (FIG. 3, right-hand side) of proBACE_{S} with both the V5 and FG epitopes indicated that none of the PCs tested could cleave this form, consistent with processing occurring at Arg₄₅. Similar results were obtained using the R42A mutant (not shown). Finally, coexpression of [BACE_{F}]_{FG} in furin-deficient LoVo cells (35) with each of the above PCs or with the yeast PC homologue kexin revealed that furin, kexin and less so PC5 could best mediate efficient intracellular processing of proBACE into BACE (not shown).

Post-translational Modifications of BACE and Their Effects on β-secretase Activity

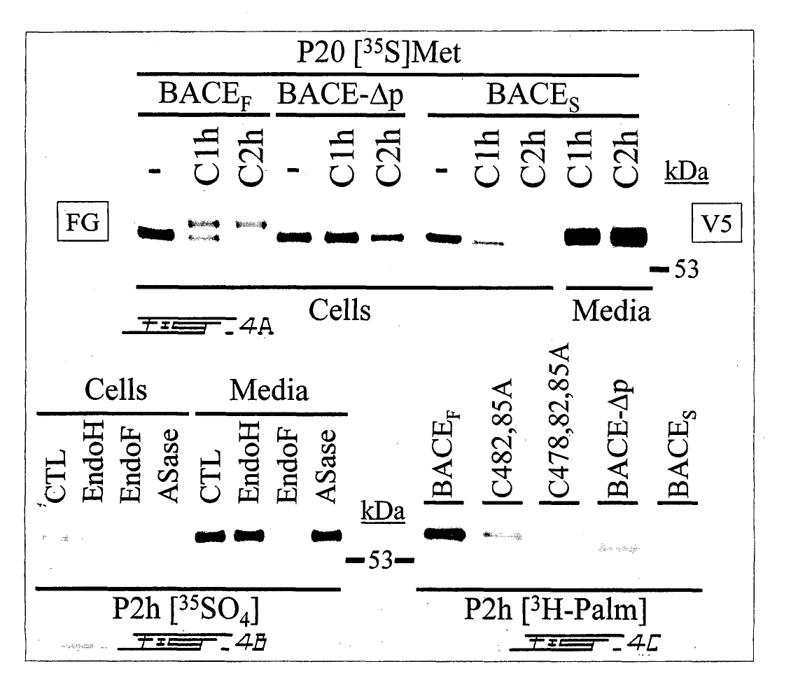

In order to investigate the functions of the prosegment and the transmembrane/cytosolic tail of BACE, a series of mutants singly tagged at the C-terminus with a FG or V5 epitope were prepared. The first construct was a truncated form of full length BACE in which the prosegment was removed (BACE-Δp). Ala mutants of three Cys residues located within the cytosolic tail of BACE_{F} that are potential Cys-linked palmitoylation sites (36) were also engineered. Accordingly, three single (Cys 478, 482 and 485) were made, as well as double (C482,485A) and triple (C478,482,485A) mutants. As previously, transiently transfected HK293 cells were pulse-labeled for 20 min with [³⁵S]Met followed by a chase of either 1 or 2h. SDS-PAGE analysis of the FG-immunoprecipitated products (FIG. 4A) revealed that, in contrast to the wild-type [BACE_{F}]_{FG}, the truncated [BACE-Δp]FG remains mostly in the ER, with only trace amounts reaching the TGN. This mutant also demonstrated a high level of endoH sensitivity and a very low level of sulfation (not shown). However, N-terminal sequencing of [³H]Arg-labeled [BACE-Δp]_{FG} revealed a major sequence with an Arg₅, indicating that the signal peptide of this mutant was poorly cleaved (not shown). These data suggest that the majority of BACE-Δp remains in the ER, and only a small fraction reaches the TGN and is sulfated. This was further corroborated by immunocytochemical evidence showing that the majority of BACE-ΔΔp immunoreactivity was concentrated in the ER (not shown). In contrast, BACE_{S} passes rapidly through the secretory pathway, as evidenced by its accumulation in the medium after 1 h of chase (FIG. 4A) and the relatively low amounts of proBACE_{S} in the ER (endoH-sensitive, lower band in cells; not shown) after either 1 or 2h of chase. By transfecting [BACE_{S}]_{FG} into HK293 cells and then labelling for 2 h with Na₂[³⁵SO₄], the intramolecular site(s) at which sulfation of BACE occurs could be examined. Equal aliquots of the FG-immunoprecipitated media were digested with endoH, endoF or aryl sulfatase (ASase). Only endoF removed the [³⁵SO₄]-label (FIG. 4B), demonstrating that sulfation occurred on one or more mature N-glycosylation sites (32), but not on tyrosine residues (33).

FIG. 4C shows the results of SDS-PAGE analysis of FG-immunoreactive proteins following a 2h labeling with [³H]palmitate of HK293 cells transiently overexpressing either BACE_{F}, its cytosolic tail Cys-mutants, BACE-Δp or BACE_{S}. Both BACE_{F} (68 kDa) and the ER-concentrated preBACE-Δp (64 kDa) were palmitoylated. When each of the three Cys residues was individually mutated, a significant decrease in the degree of palmitoylation (not shown) was observed. The double (C482,485A) mutant had ≦30% as much palmitoylation as the wild type BACE_{F}, whereas the triple mutant C478,482,485A was barely palmitoylated. The observation that each of the mutants was expressed to similar degrees based on their FG-immunoprecipitated reactivities following a 2h pulse-labeling with [³⁵S]Met was verified (not shown). These data demonstrate that palmitoylation can occur at all three of the Cys (478, 482 and 485) residues within the cytosolic tail of BACE_{F}. Predictably, soluble BACE_{S} was not palmitoylated. The fact that the 64 kDa preBACE-Δp was palmitoylated, as opposed to the mature 68 kDa BACE_{F}, suggests that this type of post-translational modification can begin at the level of the ER (36).

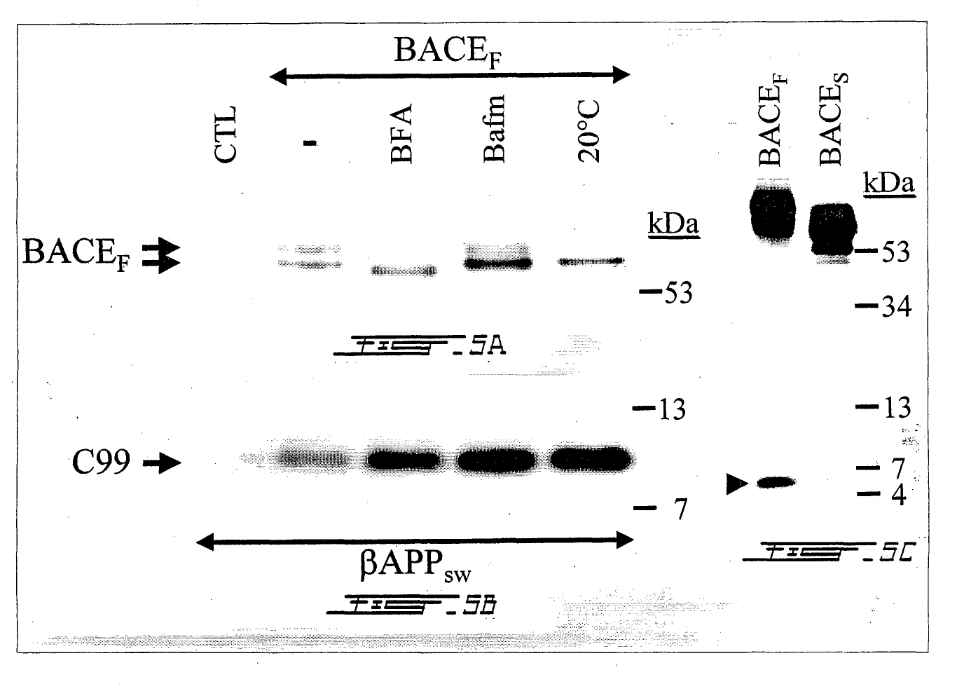

The enzymatic activity of [BACE_{F}]_{FG} was first tested in HK293 cells transfected with βAPP_{SW} cDNA. Following a 3h pulse-labeling with [³⁵S]Met (FIG. 5), the cells were exposed to either BFA, bafilomycin (an inhibitor of vesicular acidification) (37) or a 20° C. incubation (which prevents most secretory proteins from leaving the TGN) (38). FIG. 5A shows that BFA and the 20° C. incubation prevented FG-immunoprecipitated 66 kDa proBACE from escaping the ER and becoming either the 72 kDa proBACE or mature, endoH-resistant BACE (not shown), whereas bafilomycin exerted a retarding effect in the ER (compared to untreated cells). As shown in FIG. 5B, co-expression of wild-type BACE_{F} and βAPP_{SW} lead to the production of a membrane-bound ˜10 kDa intracellular product (C99) that was detected by a polyclonal antibody raised against the N-terminal 16 aa of Aβ. This band was also observed using the Aβ N-terminal-specific antibody FCA18 (23), confirming that this cleavage product began with the correct N-terminus of Aβ (starting at the β-secretase cleavage site sequence D₆₅₃AEFRHDS . . . ) (SEQ ID NO:23) and likely ended at the C-terminus of βAPP, as reported previously (11,12). Unexpectedly, regardless of the relative levels of BACE and proBACE, βAPP_{SW} was well processed in the ER. In other pulse and pulse-chase experiments it was observed that the maximal amount of C99 product was generated by BACE_{f} after a 20 min pulse, consistent with production of C99 in an early secretory compartment, likely to be the ER. Finally, tests were conducted to determine whether BACE_{F} may be transformed into a soluble shed-form. As shown in FIG. 5C, a small amount of ˜6 kDa form of FG-labeled BACE_{F} but not FG-labeled BACE_{S} could indeed be detected. This suggests that shedding of membrane-bound BACE_{F} can occur to a small extent.

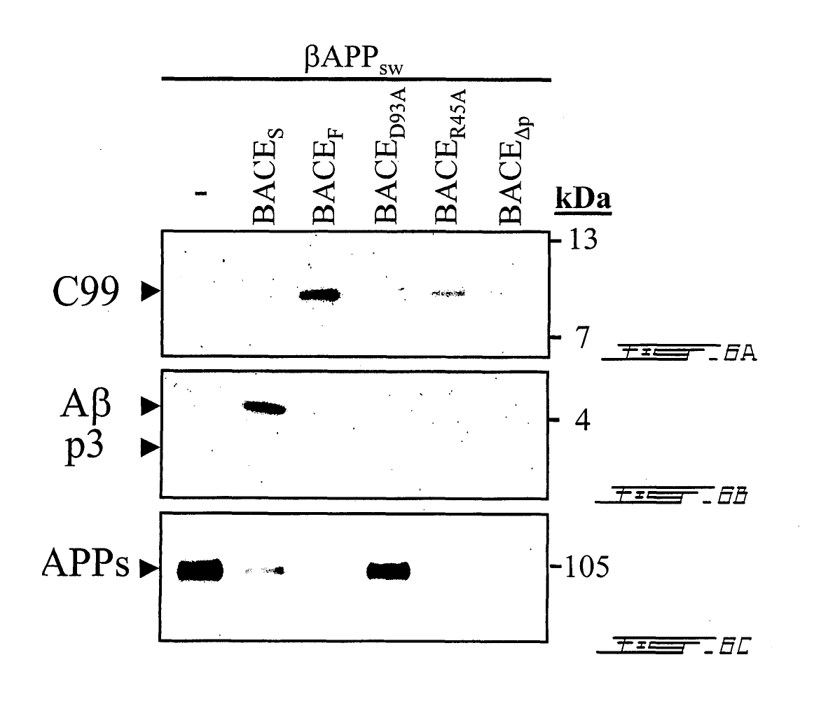

In the next set of experiments (FIG. 6), wild-type BACE and selected BACE mutants were co-expressed with βAPP_{SW}. As shown in FIG. 6A, C99 production was evident in cells co-expressing wild type BACE_{F} and βAPP_{SW} following pulse-labeling for 4h with [³⁵S]Met. Unexpectedly, the same band, although less intense, was also obtained with the mutants [BACEF-R45A] and BACE_{F}-Δp (FIG. 6A), as well as with the [BACE_{F}-R42A], [BACE_{F}-C482,485A] and [BACE_{F}-C478,482,485A] mutants (not shown), indicating that all of these isoforms have at least some activity. The absence of C99 production by the active site mutant [BACE_{F}-D93A] confirms that these activities actually correspond to BACE and its mutant forms (FIG. 6A). Notably, the soluble form of BACE_{S} produced much less C99 compared to any of the other active forms analysed, even though similar amounts of immunoreactive BACE were expressed (not shown).

Next, the secreted βAPP cleavage products were analysed using a polyclonal antibody developed against Aβ₄₀ as well as the antibody FCA3340 (not shown) recognizing the C-terminus of Aβ₄₀ (23). Both antisera recognize Aβ₄₀ (generated by the β- and γ-secretases) and Aβₓ₋₄₀ (e.g., Aβ₁₁₋₄₀ generated by overexpressed β-secretase; see ref. 11). Amazingly, BACE_{S} and, to a lesser extent, BACE-Δp were by far the forms of β-secretase that ultimately lead to the formation of the most amyloidogenic Aβ peptide (FIG. 6B). Overexpression of either BACE_{F} or BACE_{R45A} (as well as the Cys-mutants [BACE_{F}-C482,485A] and [BACE_{F}-C478,482,485A], not shown) resulted in an elevation of the level of the non-amyloidogenic Aβₓ₋₄₀ product (possibly Aβ₁₁₋₄₀, see ref. 11) with no significant change in that of Aβ₄₀. Again, as expected, [BACE_{F}-D93A] was inactive.

When the levels of secreted APP_{S} generated by α-secretase were analysed using the same 1-16 Aβ antibody, an inverse relationship between the levels of C99 and those of secreted APP_{S} was noticed. BACE_{F}, [BACE_{F}-R45A], BACE_{F}-Δp generated higher amounts of the non-amyloidogenic C99 and Aβₓ₋₄₀ along with lower levels of secreted APPs, whereas control cells or cells overexpressing the inactive [BACE_{F}-D93A] mutant secreted much more pronounced APP_{S} levels (FIG. 6C). These data provide evidence that the APP_{S} measured with the 1-16 Aβ antibody is probably APP_{S}α resulting from cleavage of βAPP by α-secretase either at the TGN or at the cell surface (5,39). In comparison, other data (FIG. 5) showed that overexpressed BACE or its mutants process βAPP_{SW} in an earlier compartment such as the ER and thus precede the action of α-secretase. Interestingly, overexpression of wild-type mouse PS1 (not shown) resulted in higher levels of either cellular C99 or secreted Aβ and APP_{S} products, suggesting that in HK293 cells wild-type PS1 increases the exposure of βAPP_{SW} to its cognate β- , α- and γ-secretases, yet does not seem to specifically increase the γ-secretase activity (40).

In order to further examine the possibility that proBACE has β-secretase activity, digestion analyses of a synthetic peptide substrate (KTEEISEVNL↓DAEFRHDSGY) (SEQ ID NO:14) encompassing the βAPP_{SW} β-secretase cleavage site were carried out in vitro using concentrated media of HK293 cells that overexpressed BACE_{S}. In four separate experiments, pre-incubation of BACE_{S}-containing media with furin produced a significant increase, 50±3%, in the level of BACE activity. In contrast, no activation of the [BACE_{S}-R45A] mutant by furin was found. Concomitant Western blotting (FIG. 3) confirmed that furin had removed the FG epitope from the prosegment of the wild-type but not the [BACE_{S}-R45A] mutant. When proBACE was affinity-purified using an anti-FLAG M1-agarose column, the resulting material had no detectable activity unless first pre-incubated with furin. These data imply that removal of the prosegment from proBACE significantly enhances the activity of this enzyme. Thus, tests were conducted to see whether a synthetic peptide representing the full-length prosegment (proBACE 22-45) would function as an inhibitor. When pre-incubated with active BACE, 20 μM of this peptide resulted in only a 20% inhibition of the Swedish peptide substrate (at 10 μM) cleavage.

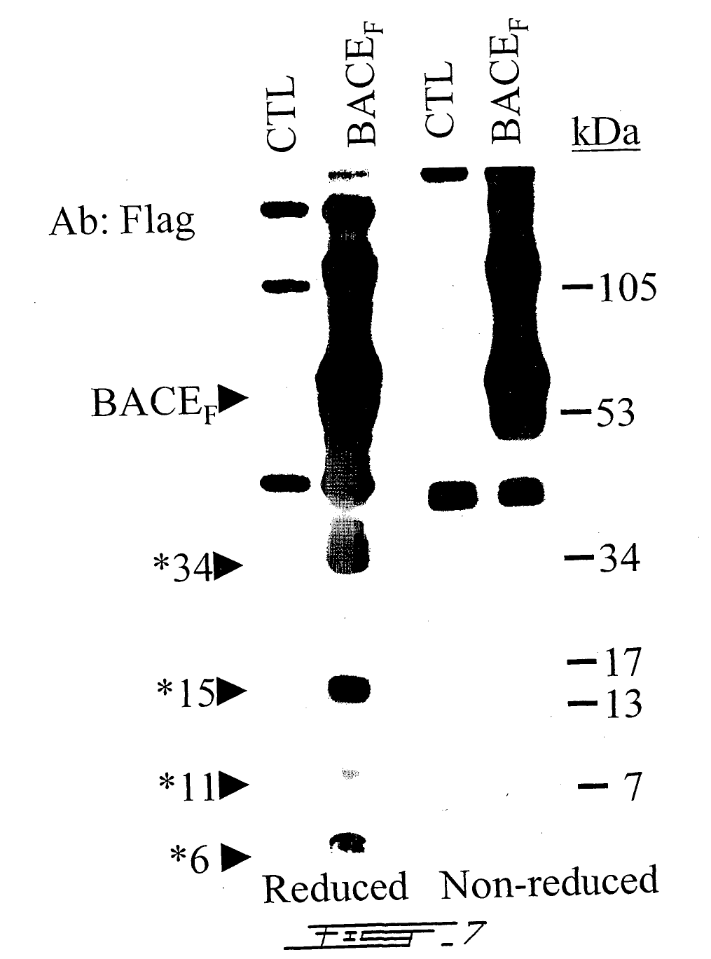

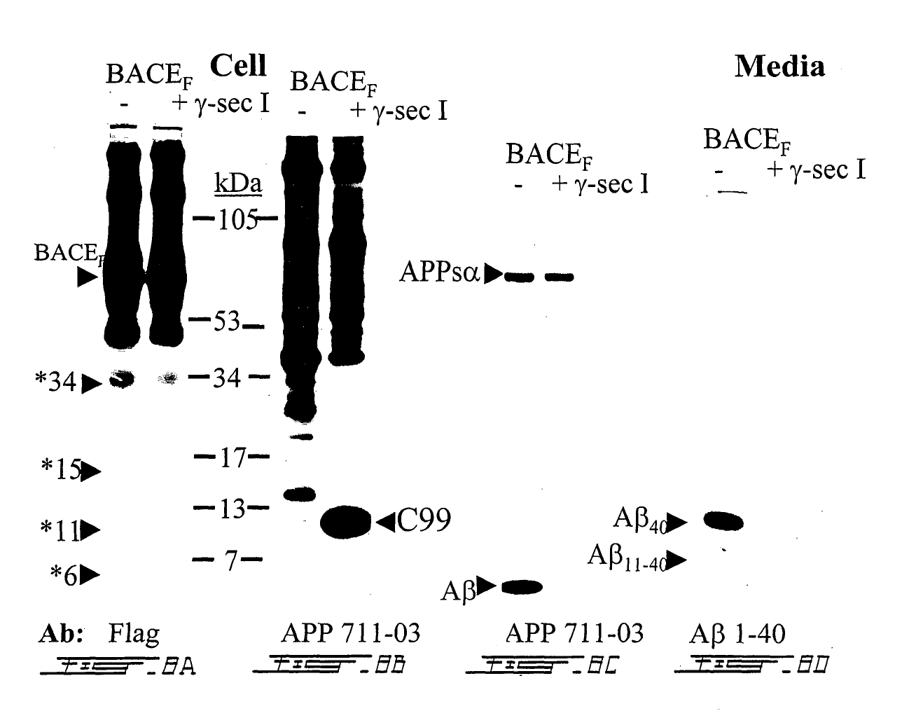

C-terminal processing of BACE—In order to further characterize the nature of apparent C-terminal cleaved BACE fragments (FIG. 5C), the analysis of [BACE_{F}]_{FG} fragments immunoprecipitated with antiserum to Flag from HK293 cells were repeated. In addition to the 6 kDa fragment noted in FIG. 5C, fragments of 34, 15, and 11 kDa are apparent (FIG. 7). Significantly, the 34 and 15 kDa bands disappear under non-reducing conditions indicating that they are disulfide linked. In addition, the intensity of the 11 and 6 kDa bands appear to diminish. It was expected that some of these BACE fragments would be disulfide linked, since it is known that the six Cys residues in the ectodomain form three intramolecular disulfide linkages (Cys²¹⁶-Cys⁴²⁰, Cys²⁷⁸-Cys⁴⁴³, Cys³³⁰-Cys³⁸⁰) (41). The 34, 15, 11 and 6 kDa BACE fragments are also apparent when [BACE_{F}]_{FG} is expressed in Neuro 2a cells (FIG. 8). The relative levels of the 11 kDa fragment compared to the other cleaved fragments of BACE appear lower in Neuro 2a compared to HK293 cells. In any case, the sites of ectodomain cleavage are apparently the same in the two cell types. Clearly, γ-secretase activity is not responsible for the formation of the 34, 15 and 11 kDa BACE fragments, since under conditions in which a γ-secretase substrate-based difluoro ketone inhibitor (46) completely inhibits AD formation (Panels C and D) and elevates cellular C99 levels (Panel B), the levels of BACE fragments are largely unchanged (Panel A). The significance of an apparent reduction in the level of the 6 kDa BACE fragment is unknown.

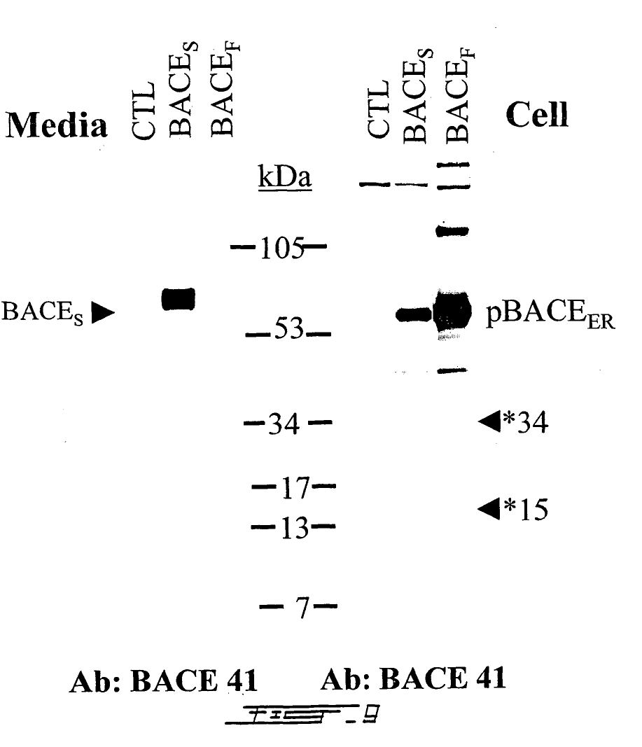

With an antiserum that recognizes a region of BACE (amino acids 186-203) that is N-terminal to both any disulfide-linked cysteines and the observed 15, 11, and 6 kDa apparent C-terminal fragments (based on size), the presence of BACE shed from BACE_{F} into the media could be detected (FIG. 9). Shed BACE appears to be smaller than BACE_{S} (truncated at Thr₄₅₄ at the lumen/extracellular border of the TM region) secreted into the media. It is noteworthy that shed BACE is larger than the major cellular form of BACE (pBACE_{ER}) due to post-translational modification. In cells, the 34 and 15 kDa truncated forms of BACE are immunoprecipated with this N-terminal antiserum as observed with the antiserum to the C-terminal flag. This result is consistent with the observation that the 34 and 15 kDa fragments of BACE are disulfide-linked (FIG. 7).

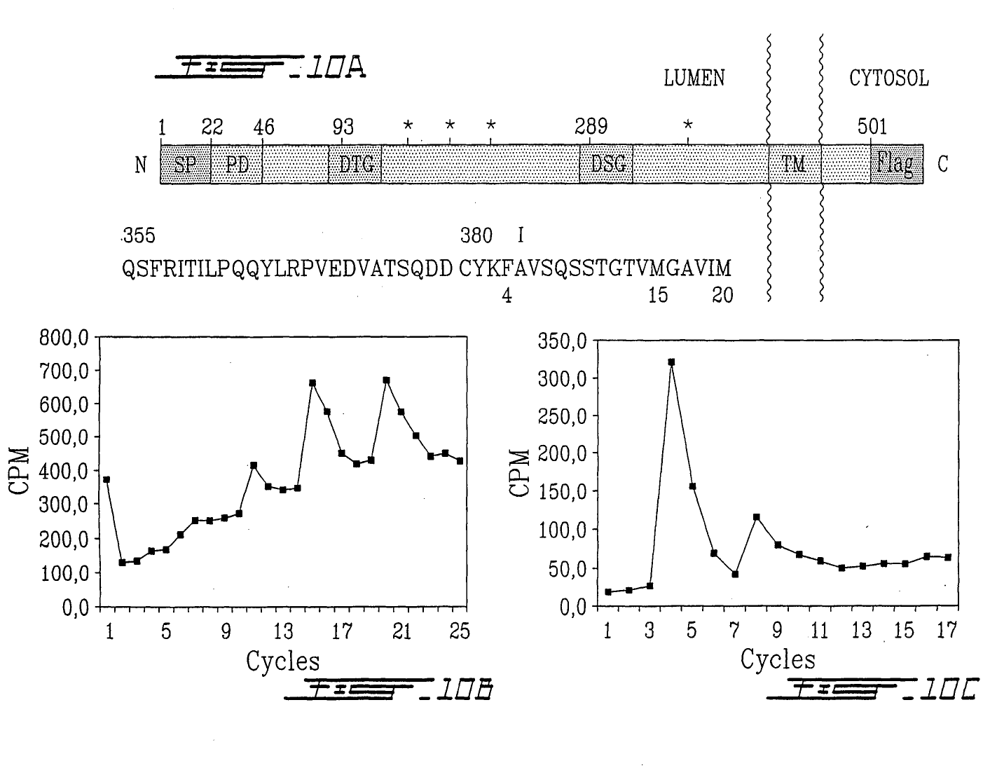

Cleavage site determination—The location of the sites of proteolytic cleavage to generate the 34, 15, 11 and 6 kDa fragments of BACE were determined by N-terminal radiosequencing of ³⁵S-Met and ³H-Phenylalanine labeled SDS-PAGE purified material. N-terminal sequence analysis of the 15 kDa BACE fragment indicated the presence of methione in positions 15 and 20, and phenylalanine in position 4 (FIG. 10). Therefore, the 15 kDa C-terminal BACE fragment starts at Cys₃₈₀ that likely results from proteolytic cleavage of BACE after Asp₃₇₉. The 34 kDa radiosequence indicates the presence of phenylalanine in position 15, which is consistent with this fragment being the N-terminus of BACE cleaved at Asp₃₇₉ (SQDD↓) (SEQ ID NO:24) with its prosegment removed by furin cleavage.

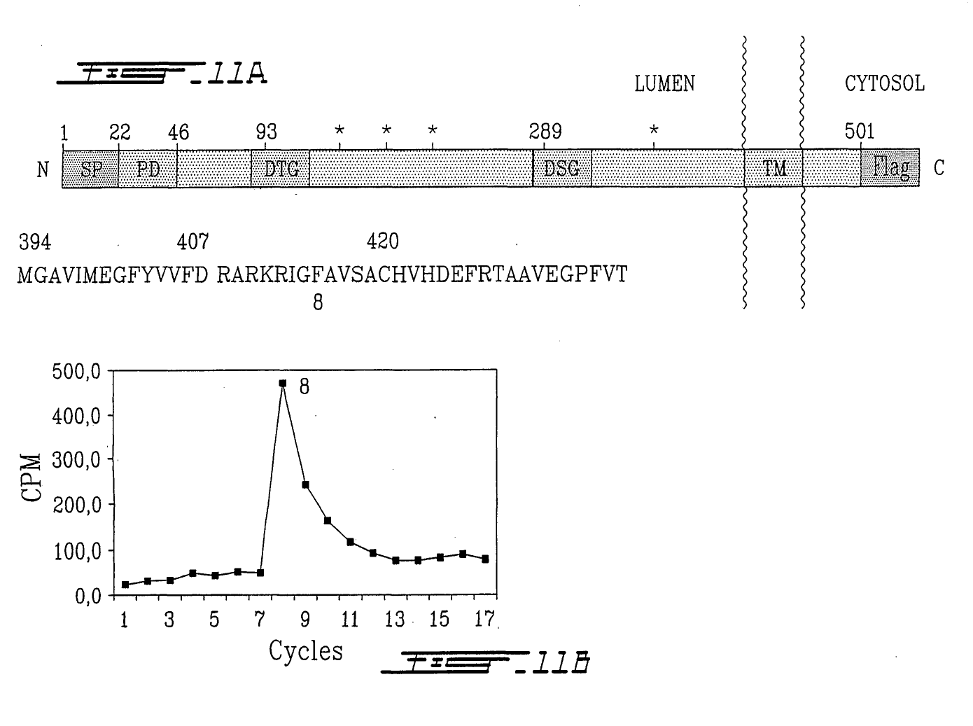

N-terminal sequence analysis of the 11 kDa fragment (FIG. 11) indicated the presence of phenylalanine in position 8 and the absence of methione. The sequence and the size of the fragment are consistent with cleavage of BACE after Asp₄₀₇ (VVFD↓) (SEQ ID NO:25). Interestingly, sequence analysis of the 6 kDa fragment indicated the presence of phenylalanine in position 8. Therefore, this fragment results from C-terminal cleavage of the 11 kDa fragment perhaps at more C-terminal Asp, likely after ASp₄₅₁(PQTD↓) (SEQ ID NO:26), in the BACE ectodomain.

Discussion

The discovery of a unique type-I membrane-bound BACE has provided a new perspective in the understanding of β-secretases (11-15). Recent data on the tissue expression of BACE in mouse and human brain (10) indicate that it co-localizes with βAPP and ADAM10 in the cortex and hippocampus of adult mice and in the cortex of human presenile patients. Furthermore, the distribution of either BACE2 or ADAM17 were not compatible with them being candidate brain β- or α-secretases, respectively.

The focus of the present work was on BACE, the more plausible β-secretase, in order to define some of its molecular and cellular trafficking properties. It was, first shown that in HK293 cells BACE is synthesized as proBACE in the ER and then moves to the TGN where it rapidly looses its prosegment due to cleavage by an α1-PDX inhibitable convertase(s). Next, it was shown that, aside from α1-PDX and the furin-site mutated α2-macroglobulin, other inhibitors such as the preprosegments of furin and PC5 can also inhibit proBACE processing. This cleavage occurs at the sequence R₄₂LPR₄₅↓ of proBACE sulfated at one or more of its carbohydrate moieties. Since sulfation of sugars occurs in the TGN (32) and PCs, except perhaps PC5-B (34), are active only in this compartment or beyond, these were taken as indications that processing of proBACE to BACE occurs in the TGN or in post TGN-vesicles. In vitro digestion of proBACE (FIG. 3) and ex vivo co-expression of BACE and the PCs in the furin-negative LoVo cells (not shown) demonstrated that zymogen processing was best performed by furin, and less so by PC5.

Next, the data generated showed that full length BACE_{F} is palmitoylated at the cytosolic tail cysteines 478, 482 and 485 and that a soluble form of BACE_{S} is not (FIG. 4C). Interestingly, BACE_{S} seems to be rapidly secreted from and does not accumulate within the cell, suggesting that the cytosolic segment of BACE_{F} must contain determinants that control cellular trafficking rates and destination. One such element could be Cys-palmitoylation, since pulse-chase experiments demonstrated that the triple mutation C478,482,485A results in slowing down exit of proBACE from the ER (not shown). However, immunocytochemical analysis of the localization of [BACE_{F}]_{FG} and [BACE_{F}-C478,482,485A]_{FG} failed to reveal gross differences in their cellular distribution (not shown). Although the role of palmitoylation of BACE, which begins in the ER, remains to be elucidated, this modification may provide a second anchor to the plasma membrane, thus directing the protein to discrete membrane microdomains or remodeling the structure of its cytoplasmic region (36).

Mutagenizing either of the arginines found to be critical for the prosegment removal, i.e., R42A or R45A, did not result in significant alteration of the trafficking rate of proBACE to the TGN, as estimated by pulse-chase (FIG. 2A) and sulfation rate analyses. At around the same time as the present results were coming to light, two in press reports on the biosynthesis of BACE reported similar observations (41,42). In the report by Capell et al. regarding the prosegment removal of human BACE (42), their data, like the present results, also revealed that such processing occurs in the TGN and that BACE_{S} trafficks more rapidly than BACE_{F} towards the TGN. The data differ from theirs, which suggests that the R45A mutant of human BACE does not exit the ER. The triplicate pulse-chase data (FIG. 2A) clearly demonstrate that the exit of both proBACE_{F} and proBACE_{F}-R45A (or R42A) to the TGN is slow but does in fact occur to a similar extent for both forms.

An interesting observation was made when the rate of exit of proBACE from the ER was analysed at 20° C., a temperature which normally blocks the budding of TGN vesicles, but which should not prevent movement from the ER to the TGN (38). Amazingly, at 20° C. proBACE cannot exit the ER, as is the case with BFA and, much less so, bafilomycin treatments (FIG. 5A). This is reminescent of the observation that αβ integrins do not exit the ER at 20° C. because of their inablity to heterodimerize (43). Whether this means that BACE is part of a larger complex, such as the one involving presenilins/γ-secretase (44), is not yet clear. It was previously reported that the production of Aβ₄₀ and Aβ₄₂ was abrogated at 20° C. (17). The present data show that proBACE can process βAPP_{SW} into C99 in the ER (FIG. 5B), suggesting that γ-secretase activity could be the limiting factor at 20° C. Even though the holoenzymes BACE and proBACE (not shown) exhibit an in vitro pH optimum of 4.5 for cleavage of synthetic peptides mimicking the β-site (11,12,15), the present data is strongly suggestive of the presence of active BACE within the neutral pH environment of the ER (FIG. 5B). The in vitro data further showed that removal of the prosegement by furin maximizes the activity of BACE. The combined observations that the active-site mutant [BACE_{F}-D93A] can lose its prosegment (not shown), that BACE did not cleave the PC-cleavage site spanning peptide (aa 39-58 of BACE), and that PCs such as furin and PC5 can remove the prosegment of BACE in vitro and ex vivo support the notion that BACE does not autoactivate, but likely requires a furin-like enzyme for zymogen activation. Alternatively, the possibility that there are other enzymes or proteins that can interact with proBACE and activate it by cleavage or dislocation of its prosegment cannot be ruled out. Indeed, experiments using affinity-purified BACE indicated that furin-treated BACE is much more active than proBACE. The finding that the BACE zymogen is apparently active is reminescent of observations regarding the processing of the relatively inactive prorenin to renin by PC5 (45). Modeling of mouse proBACE based on the structure of a close homologue human proGastricsin suggested that the prosegment acts as a flap covering the active site of BACE and that the furin-processing site R₄₂-X-X-R₄₅↓ is quite accessible to cleavage (not shown).

In an effort to define the importance of cellular trafficking on the production of C99 and Aβ, the ability of various engineered forms of BACE to process βAPP_{SW} and ultimately to generate amyloidogenic peptides was compared. Surprisingly, overexpression of the soluble form of BACE_{S} results in a very significant increase in the levels of secreted Aβ (FIG. 6B). This experiment, which was repeated 4 times, suggests that the rapid trafficking of the soluble form through the TGN and at the cell surface may favor the production of C99 in a microcompartment close to where γ-secretase is active. An exciting extension of this model would be that the amyloidogenic potential of BACE is enhanced by BACE C-terminal processing by BACE secretase/sheddase. In both HK293 and Neuro2a cells 34, 15, 11 and 6 kDa BACE fragments (FIGS. 7 and 8) and BACE shed into the media (FIG. 9) as the result of BACE secretase/sheddase activity were detected. Finally, overexpression of the active site mutant [BACE_{F}-D93A] in N2a cells stably overexpressing βAPP_{SW} (17) did not affect the generation of either C99 or Aβ by endogenous secretases (not shown), suggesting that this mutant cannot act as a dominant negative, as was the case for the active site mutant of the candidate α-secretase ADAM10(5).

Thus, the results reported above reveal that BACE can process βAPP_{SW} in the ER and that furin or PC5 process the zymogen in the TGN, possibly in order to maximize its activity in acidic cellular compartments. BACE undergoes a number of other post translational modifications such as carbohydrate sulfation and cytosolic tail Cys-palmitoylation which may finely regulate its rate of trafficking and cellular destination(s). The in vivo physiological function of BACE remains to be elucidated as well as the possibility that this enzyme may be part of a larger complex with other proteins, including the other secretases involved in the processing of βAPP.

BACE Secretase/Sheddase Activity—In addition to the data reported above, a novel proteolytic activity that cleaves the ectodomain (juxtamembrane region on the lumen/extracellular side) of BACE after Asp₃₇₉ (SQDD↓) (SEQ ID NO:24) and Asp₄₀₇ (VVFD↓) (SEQ ID NO:25), and likely after Asp₄₅₁(PQTD↓) (SEQ ID NO:26) has been identified (FIGS. 10 and 11). This activity has been identified as BACE secretase/sheddase. The shed form of BACE (FIG. 9) most likely results from cleavage after Asp₄₅₁(PQTD↓), since it is the only juxtamembrane Asp C-terminal to Cys₄₄₃ that is reported to be linked via a disulfide to Cys₂₇₈ (41). The data indicate that the 15 kDa Asp₃₇₉ cleavage product, and to some extent the 11 kDa Asp₄₀₇ cleavage product, are disulfide linked (FIG. 7).

A diverse set of transmembrane proteins are known to undergo proteolysis in their juxtamembrane regions leading to the release of their extracellular domains into the surrounding milieu (reviewed in 47-49). This process, which has been termed ectodomain shedding, affects a wide variety of proteins, including cytokines, growth factors and their receptors, and adhesion molecules. The unusual P1 Asp-ase activity of BACE secretase/sheddase has not been observed in other cases of ectodomain shedding.

Based on inhibitor studies, ectodomain shedding is predominantly mediated by metalloproteases. Specifically, several members of the ADAM family of metalloproteases (a disintegrin and metalloprotease) have been implicated as ectodomain sheddases (reviewed in 50,51). For example, Kuzbanian (Kuz, ADAM 10) can cleave the Notch ligand Delta and has been shown to have APP α-secretase activity (5). In addition to the ADAM proteases, at least one matrix metalloprotease, MMP-7 (matrilysin) has a functionally relevant role in shedding (52,53). A recent report, suggests that the metalloproteases Meprin A and B can function as sheddases (54). The metalloprotease inhibitors GM6001 (Chemicon International) and TAPI-1 (Peptides International) did not inhibit BACE secretase/sheddase activity in Neuro 2a cells. In a few cases, serine proteases such as proteinase 3 (55) and a putative chymotrypsin-like protease (56) appear to be the enzymes responsible for ectodomain shedding.

The distance of cleavage in BACE from the membrane by BACE secretase/sheddase varies from 5, 48 to 76 amino acids for cleavage after Asp₄₅₁(PQTD↓) (SEQ ID NO:26), Asp₄₀₇ and Asp₃₇₉ (SQDD↓) (SEQ ID NO:24) respectively. In other cases of ectodomain shedding, this distance varies with the substrate and protease class ranging from intramembranous to 93 amino acids, with the majority of ectodomain shedding resulting from cleavage between 12 to 24 amino acids from the membrane (reviewed in 48).

Ectodomain shedding may occur in an intracellular compartment. For example, ADAM-mediated ectodomain shedding by at least two family members, tumor necrosis factor α convertase (TACE) and ADAM 10 may occur in an intracellular compartment in addition to the cell surface (5,57). Intracellular ectodomain shedding may occur by a process recently called Regulated intramembrane proteolysis (Rip)(57). Rip has been shown to occur during the processing of mammalian proteins (e.g. SREBP, Notch, Ire1 and ATF6). For example, SREBP cleavage occurs at a leucine/cysteine bond, three residues into the hydrophobic/transmembrane segment (58,59). Another example of RIP, is the aspartyl protease inhibitor dependent γ-secretase cleavage of APP by a protein complex containing presenilin 1 and presenilin 2(60). This apparent intramembranous cleavage of the Aβ40-41 and Aβ42-43 peptide bonds within C99 and C83 generates Aβ40 and Aβ42 and p3-40 and p3-42 (reviewed in 61). Clearly, γ-secretase differs from BACE secretase/sheddase since a substrate-based difluoro ketone inhibitor does not inhibit the later (FIG. 8).

The unusual P1 Asp-ase activity of BACE secretase/sheddase is similar to that reported for members of the caspase (cysteinyl-directed aspartate-specific protease) family and the T-lymphocyte serine protease granzyme B (reviewed in 62-64). However, these enzymes cleave their substrates in the cytoplasm or on the cytoplasmic side of organelles. For example, caspase-12 associated with the ER and caspase 2 associated with Golgi cleave substrates on the cytoplasmic surface (65,66). Granzyme B, although secreted from cytotoxic secretory granules, cleave pro-caspases and other substrates in the cytoplasm of target cells (64). The nonselective pancaspase inhibitor Z-Val-Ala-Asp(OMe)-CH₂F (Calbiochem) at 100 uM, a concentration which inhibits the majority of caspases (67), had no effect on the BACE secretase/sheddase activity in Neuro 2a cells.

Although the present invention has been described hereinabove by way of preferred embodiments thereof, it can be modified without departing from the spirit and nature of the subject invention as defined in the appended claims.

### LIST OF REFERENCES

1. Selkoe D. J. (1997) Science 275:630-631.

2. Naslund J., Haroutunian V., Mohs R., Davis K. L., Davies P., Greengard P., and Buxbaum J. D. (2000) JAMA 283, 1571-1572.

3. Checler F. (1995) J. Neurochem. 65, 1431-44.

4. Buxbaum J. D., Liu K.-N., Luo Y., Slack J. L., Stocking K. L., Peshon J. J., Johnson R. S., Castner B. J., Cerretti D. P., and Black R. A. (1998) J. Biol. Chem. 273, 27765-2767.

5. Lammich S., Kojro E., Postina R., Gilbert S., Pfeiffer R., Jasionowski M., Haass C., and Fahrenhol F. (1999) Proc. Natl. Acad. Sci. USA 96, 3922-3927.

6. Koike, H., Tomioka, S,. Sorimachi, H., Saido, T. C., Maruyama K., Okuyama A., Fujisawa-Sehara A., Ohno, S., Suzuki K., and Ishiura, S. (1999) Biochem. J. 343, 371-375.

7. Seidah, N. G. and Chrétien, M. (1999) Brain Res. 848, 45-62.

8. Anderson, E. D., Thomas, L., Hayflick, J. S. and Thomas, G. (1993) J. Biol. Chem., 268, 24887-24891

9. Lopez-Perez E., Seidah N. G., and Checler F. (1999) J Neurochem. 73:2056-2062.

10. Marcinkiewicz, M, and Seidah, N. G. (2000) J. Neuroclem. (in press).

11. Vassar R., Bennett B. D., Babu-Khan S., Kahn S., Mendiaz E. A., Denis P., Teplow D. B., Ross S., Amarante P., Loeloff R., Luo Y., Fisher S., Fuller J., Edenson S., Lile J., Jarosinski M. A., Biere A. L., Curran E., Burges T., Louis J.-C., Collins F., Treanor J., Rogers G., and Citron M. (1999) Science 286, 735-741.

12. Hussain I., Powell D., Howwlett D. R., Tew D. G., Meek T. D., Chapman C., Gloger I. S., Murphy K. E., Southan C. D., Ryan D. M., Smith T. S., Simmons D. L., Walsh F. S., Dingwall C., and Christie G. (1999) Mol. Cell. Neurosci. 14,419-427.

13. Sinha S., Anderson J. P., Barbour R., Basi G. S., Caccavello R., Davis D., Doan M., Dovey H. F., Frigon N., Hong J., Jacobson-Croak K., Jewett N., Keim P., Knops J., Lieberburg I., Power M., Tan H., Tatsuno G., Tung J., Schenk D., Seubert P., Suomensaari S. M., Wang S., Walker D., John V., Zhao J., McConlogue L., and John V. (1999) Nature 402, 537-540.

14. Yan R., Bienkowski M., Shuck M. E., Miao, H., Tory M. C., Pauley A. M., Brashier J. R., Stratman N. C., Mathews W. R., Buhl A. E., Carter D. B., Tomasselli A. G., Parodi L. A., Heirikson R. L., and Gurney M. E. (1999) Nature 402, 533-537.

15. Lin X., Koelsch G., Wu S., Downs D., Dashti A., and Tang J. (2000) Proc. Natl. Acad. Sci. USA 97,1456-1460.

16. Sauder, J. M., Arthur, J. W., and Dunbrack, R. L. Jr (2000) J. Mol. Biol. 300, 241-248.

17. Greenfield, J. P., Tsai, J., Gouras, G. K., Hai, B., Thinakaran, G., Checler, F., Sisodia, S. S., Greengard, P., Xu, H.(1999) Proc. Natl. Acad. Sci. USA 96, 742-747.

18. Van Gassen, G., Annaert, W. and Van Broeckhoven, C. (2000) Neurobiol. Disease 7, 135-151.

19. Wolfe, M. S., Xia, W., Moore, C. L., Leatherwood, D. D., Ostaszewski, B., Rahmati, T., Donkor, I. O., and Selkoe, D. J. (1999) Biochemistry 38, 4720-4727.

20. van de Loo, J. W., Creemers, J. W., Bright, N. A., Young, B. D., Roebroek, A. J., and Van de Ven, W. J. (1997) J. Biol. Chem. 272,27116-27123

21. Benjannet, S., Savaria, D., Laslop, A., Munzer, J. S., Chrétien, M., Marcinkiewicz, M. and Seidah, N. (1997) J. Biol. Chem. 272, 26210-26218.

22. Sisodia, S. S., Koo, E. H., Beyreuther, K., Unterbeck, A., and Price, D. L. (1990) Science 248, 492-495.

23. Barelli, H., Lebeau, A., Vizzavona, J., Delaere, P., Chevallier, N., Drouot, C., Marambaud, P., Ancolio, K., Buxbaum, J. D., Khorkova, O., Heroux, J., Sahasrabudlie, S., Martinez, J., Warter, J.-M., Mohr, M. and Checler, F. (1997) Mol. Medicine 3, 695-707.

24. Zhong, M., Munzer, J. S., Basak, A., Benjannet, S., Mowla, S. J., Decroly, E., Chrétien, M. and Seidah, N. G. (1999) J. Biol. Chem. 274, 33913-33920.

25. Lusson, J., Vieau, D., Hamelin, J., Day, R., Chrétien, M. and Seidah, N. G. (1993) Proc. Natl. Acad. Sci. USA 90, 6691-6695.

26. Seidah, N. G., Mowla, S. J., Hamelin, J., Mamarbachi, A. M., Benjalnet, S., Toure, B. B., Basak, A., Munzer, J. S., Marcinkiewicz, J., Zhong, M., Barale, J. C., Lazure, C., Murphy, R. A., Chrétien, M., and Marcinkiewicz, M. (1999) Proc. Natl. Acad. Sci. USA 96,1321-1326.

27. Touré, B. B., Basak, A., Munzer, J. S., Benjannet, S., Rochemont, J., Lazure, C., Chrétien, M. and Seidah, N. G. (2000) J. Biol. Chem. 275, 2349-2358.

28. Rompaey, L. V., Ayoubi, T., Van De Ven, W., and Marynen, P. (1997) Biochem. J. 326, 507-514.

29. Decroly, E., Wouters, S., Dibello, C., Lazure, C., Ruysschaert, J. M. and Seidah, N. G. (1996) J. Biol. Chem. 271, 30442-30450.

30. Munzer, J. S., Basak, A., Zhong, M., Mamarbachi, A., Hamelin, J., Savaria, D., Lazure, C., Benjannet, S., Chrétien, M. and Seidah, N. G. (1997) J. Biol. Chem. 272, 19672-19681.

31. Lippincott-Schwartz J., L. Yuan, C. Tipper, M. Amherdt, L. Orci, and R. D. Klausner (1991) Cell 67, 601-616.

32. Karaivanova, V. K., and Spiro, R. G. (1998) Biochem. J. 329, 511-518.

33. Beisswanger, R., Corbeil, D., Vannier, C., Thiele, C., Dohrmann, U., Kellner, R., Ashman, K., Niehrs, C., Huttuer, W. B. (1998) Proc. Natl. Acad. Sci. USA 95, 11134-11139.

34. Xiang, Y., Molloy, S. S., Thomas, L., and Thomas, G. (2000) Mol. Biol. Cell 11, 1257-1273.

35. Takahashi, S., Nakagawa, T., Kasai, K., Banno, T., Duguay, S. J., Van, V., Murakami, K. and Nakayama, K. (1995) J. Biol. Chem. 270, 26565-26569.

36. Schweizer, A., Kornfeld, S., Rohrer, J. (1996) J. Cell Biol. 132, 577-584.

37. Bowman, E. J., Siebers, A. and Altendorf, K. (1988) Proc. Natl. Acad. Sci. USA 85, 7972-7976.

38. Matlin, K. S., and Simons, K. (1983) Cell 34, 233-243.

39. Skovronsky, D. M., Moore, D. B., Milla, M. E., Doms, R. W., and Lee, V. M. (1999) J. Biol. Chem. 275, 2568-2575.

40. da Costa, C. A., Ancolio, K., and Checler, F. (1999) Mol., Med. 5, 160-168.

41. Haniu, M., Denis, P., Young, Y., Mendiaz, E. A., Fuller, J., Hui, J. O., Bennett, B. D., Kahn, S., Ross, S., Burgess, T., Katta, V., Rogers, G., Vassar, R. and Citron, M. (2000) J. Biol. Chem 275, 21099-21106.

42. Capell, A., Steiner, H., Willem, M., Kaiser, H., Meyer, C., Walter, J., Lammich, S., Multhaup, G., and Haass C. (2000) J. Biol. Chem. PMI: 10801872

43. Rigot, V.; Andre, F.; Lehmann, M.; Lissitzky, J. C.; Marvaldi, J.; Luis, J. (1999) Eur. J. Biochem. 261, 659-666.

44. Yu, G., Chen, F., Levesque, G., Nishimura, M., Zhang, D. M., Levesque, L., Rogaeva, E., Xu, D., Liang, Y., Duthie, M., St George-Hyslop, P. H., and Fraser, P. E. (1998) J. Biol. Chem. 273, 16470-16475.

45. Mercure, C., Jutras, I., Day, R., Seidah, N. G., and Reudelhuber, T. L. (1996) Hypertension 28, 840-846.

46. Wolfe, M. S. et al. (1998) J. Med. Chem. 41, 6-9.

47. Blobel, C. P. (2000) Curr Opin Cell Biol 12 (5), 606-612.

48. Hooper, N. M., Karran, E. H., and Turner, A. J. (1997) Biochem J. 321, 256-279.

49. Massague, J., and Pandiella, A. (1993) Annu Rev Biochem 62, 515-541.

50. Turner, A. J., and Hooper, N. M. (1999) Biochem Soc Trans 27 (2), 255-259.

51. Black, R. A., and White, J. M. (1998) Curr Opin Cell Biol 10 (5), 654-659.

52. Haro, H. et al. (2000) J Clin Invest 105 (2), 143-150.

53. Powell, W. C. et al. (1999) Curr Biol 9 (24), 1441-1447.

54. Bertenshaw, G. P. et al. (2001) J. Biol. Chem. (In press).

55. Coeshott, C. et al. (1999) Proc Natl Acad Sci USA 96 (11), 6261-6266.

56. Lee, M. C. et al. (2000) Biochem. Biophys. Res. Commun. 279, 116-123.

57. Schlöndorff, J. et al. (2000) Biochem. J. 347, 131-138.

58. Ye, J. et al. (2000) PNAS 97, 5123-5128.

59. Brown, M. S. et al. (2000) Cell 100, 391-398.

60. Yu, G. et al. (2000) Nature 407, 48-54.

61. Selkoe, D. J. (1999) Nature 399, A23-A31.

62. Wellington, C. L. and Hayden, M. R. (2000) Clin. Genet. 57, 1-10.

63. Kidd, V. J., Lahti, J. M., and Teitz, T. (2000) seminars in Cell & Developmental Biology, Vol. 11, pp. 191-201.

64. Kam, C.-M., Hudig, D., and Powers, J. C. (2000) Biochimica et Biophysica Acta 1477, 307-323.

65. Nakagawa, T. amd Yuan, J. (2000) The Journal of Cell Biology 150, 887-894.

66. Mancini, M. et al. (2000) J. Cell Biol. 149, 603-612.

67. Roy, S. and Nicholson, D. W. (2000) Methods in Enzymology 322, 110-125.

What is claimed is:

1. A secretase/sheddase which is characterized by having Asp-ase activity on a beta-site APP-cleaving enzyme but which is not a member of the caspase family.

2. A secretase/sheddase as defined in claim 1, wherein said beta-site APP-cleaving enzyme is BACE, Asp2 or memepsin 2.

3. A secretase/sheddase as defined in claim 2, wherein said beta-site APP-cleaving enzyme is BACE.

4. Use of an inhibitor of a secretase/sheddase as defined in claim 3 in the making of a medication for preventing cleavage of BACE.

5. A use as defined in claim 4, wherein said inhibitor is selected from the group consisting of:
a ribozyme that specifically targets and degrades BACE secretase/sheddase mRNA, a peptide that interferes with the binding of BACE secretase/sheddase with BACE, an antibody that functions as an inhibitor of BACE secretase/sheddase activation, and an antagonist that functions as an inhibitor of BACE secretase/sheddase activation.

6. A use as defined in claim 5, wherein said inhibitor is an Asp-ase inhibitor.

7. A use as defined in claim 5 or 6 for the treatment of a neurodegenerative disorder that is characterized by the generation of Aβ protein.

8. A use as defined in claim 7, wherein said neurodegenerative disorder is Alzheimer's Disease.

9. Use of an agent selected from the group consisting of
a ribozyme that specifically targets and degrades BACE secretase/sheddase mRNA, a peptide that interferes with the binding of BACE secretase/sheddase with RACE, an antibody that functions as an inhibitor of BACE secretase/sheddase activation, an antagonist that functions as an inhibitor of BACE secretase/sheddase activation, an agonist that functions as an activator of BACE secretase/sheddase
to produce a medicament for therapeutically modulating the activity of a secretase/sheddase as defined in claim 3.

10. A use as defined in claim 9, wherein said modulation consists in the inhibition of a secretase/sheddase as defined in claim 3 to prevent cleavage of BACE.

11. A use as defined in claim 10, wherein said agent is an Asp-ase inhibitor.

12. A method for the identification of an agent that can alter the ability of a secretase/sheddase as defined in claim 3 to associate with and process a known substrate, comprising:
in a reaction mixture, allowing said BACE secretase/sheddase to bind to said known substrate of said BACE secretase/sheddase in the presence of an agent to be tested; and
measuring the production of BACE C-terminal cleavage products, shed BACE or AA in the presence of said agent to be tested, and comparing same under conditions when said agent to be tested is absent from the reaction mixture.

13. A method as defined in claim 12, wherein said known substrate is BACE, BACE fragments, or the indirect substrate βAPP.

14. A method as defined in claim 13, wherein said known substrate is labeled with a detectable moiety.

15. A method as defined in claim 14, wherein said detectable moiety is a radionuclide, an antibody or fluorescent label.

16. A method as defied in any one of claims 12-15, which is automated.

17. Use of a method as defined in claim 16 for high throughput screening of agents.

18. A method for identifying a candidate compound that modulates BACE secretase/sheddase biological activity in vitro, said method comprising the steps of:
i) providing BACE secretase/sheddase and a BACE secretase/sheddase substrate;
ii) contacting said BACE secretase/sheddase and BACE secretase/sheddase substrate with a candidate compound; and
iii) measuring the biological activity of said BACE secretase/sheddase,
wherein a change in the biological activity of BACE secretase/sheddase relative to the absence of a candidate compound indicates a candidate compound that modulates BACE secretase/sheddase biological activity.

19. The method of claim 18, wherein said BACE secretase/sheddase and said BACE secretase/sheddase substrate are derived from an intracellular compartment.

20. The method of claim 19, wherein said intracellular compartment further comprises amyloid precursor protein (APP).

21. The method of claim 18, wherein said biological activity of said BACE secretase/sheddase comprises the cleavage of a BACE secretase/sheddase substrate.

22. The method of claim 20, wherein said biological activity of said BACE secretase/sheddase comprises the cleavage of APP at the beta cleavage site.

23. The method of claim 18, wherein said method is performed in the absence of a membrane permeabilizing reagent.

24. The Method of claim 18, wherein said BACE secretase/sheddase substrate is selected from a group consisting of BACE, a fragment of BACE containing amino acids asp³⁷⁹, asp⁴⁰⁷, or asp⁴⁵¹, SEQ ID NO: 27, and SEQ ID NO: 28.

25. The method of claim 18, wherein said method is performed in the presence of a protease inhibitor.

26. The method of claim 25, wherein said protease inhibitor is selected from a group consisting of panCaspase inhibitors, GM 6001, TAPI-1, serine protease inhibitors, and γ-secretase difluoro ketone inhibitor.

27. The method of claim 18, wherein said modulator of BACE secretase/sheddase biological activity is an inhibitor of said biological activity.

28. A substantially pure polypeptide or analog thereof having the amino acid sequence set forth in SEQ ID NO: 27, SEQ ID NO: 28, or amino acids 407-456 of SEQ ID NO: 19, or a fragment of said polypeptide, wherein said fragment is a substrate of BACE secretase/sheddase.

29. A method for treating a neurodegenerative disorder, said method comprising administering a therapeutic amount of a pharmaceutical composition comprising a polypeptide set forth in claim 28.

30. The method of claim 29, wherein said neurodegenerative disorder is Alzheimer's disease.

31. The polypeptide of claim 28, wherein said polypeptide is detectably labeled.

32. The polypeptide of claim 31, wherein said label is a fluorescent tag or a radionuclide.

33. The polypeptide of claim 28, wherein said polypeptide is resistant to proteolysis at amino acid asp³⁷⁹, asp⁴⁰⁷, or asp⁴⁵¹.

34. An isolated polypeptide complex comprising amino acids 46-451 of SEQ ID NO: 19, wherein said amino acids 46-451 are cleaved at any one of amino acids asp³⁷⁹, asp⁴⁰⁷, and asp⁴⁵¹, and wherein said cleaved polypeptides are linked by intramolecular disulphide bonds.

35. A method of determining whether an individual is at risk of developing a neurodegenerative disorder that is characterized by the generation of Aβ protein, comprising:
providing a tissue or fluid sample from said individual;
reacting said tissue or fluid sample with a secretase/sheddase as defined in claim 3; and
determining whether the level of BACE C-terminal cleavage products, shed RACE or Aβ in said sample is higher than the level in a sample of a healthy subject, as an indication that the individual is at risk of developing a neurodegenerative disorder that is characterized by the generation of Aβ protein.

36. A method as defined in claim 35, wherein said tissue or fluid sample is cerebrospinal fluid (CSF) or blood platelets.

37. A method as defined in claim 35 or 36, wherein said neurodegenerative disorder is Alzheimer's Disease.

38. A kit comprising a container or containers comprising a secretase/sheddase as defined in claim 3 and at least one substrate selected from the group consisting of BACE, BACE fragments, or the indirect substrate βAPP.
